(19) United States
(12) Patent Application Publication
(10) Pub. No.: US 2025/0027033 A1
(43) Pub. Date: Jan. 23, 2025

(54) MODIFIED STRAINS OF CHLORELLA VULGARIS AND METHOD OF PRODUCTION

(71) Applicant: ALGENUITY HOLDINGS LTD, Stewartby Bedfordshire (GB)

(72) Inventors: Andrew Pearce SPICER, STEWARTBY Bedfordshire (GB); Alexander Findlay PUDNEY, STEWARTBY Bedfordshire (GB); Richard Thomas SMITH, STEWARTBY Bedfordshire (GB); Jean-Charles Eric Francois ISNER, STEWARTBY Bedfordshire (GB); Gino SCHIANO DIVISCONTE, STEWARTBY Bedfordshire (GB)

(73) Assignee: ALGENUITY HOLDINGS LTD, Stewartby Bedfordshire (GB)

(21) Appl. No.: 17/999,955

(22) PCT Filed: Nov. 19, 2020
(86) PCT No.: PCT/IB2020/060941

§ 371 Date: Nov. 28, 2022

(30) Foreign Application Priority Data
May 27, 2020 (GB) 2007924.0

(51) Int. Cl. C12N 1/12; A23L 29/00; A61K 8/9722; C12N 9/00; C12N 9/02; C12N 9/10; C12N 15/01; C12R 1/89

(52) U.S. Cl. CPC C12N 1/12; A23L 29/065; A61K 8/9722; C12N 9/001; C12N 9/1007; C12N 9/93; C12N 15/01; C12R 2001/89; C12Y 103/05005; C12Y 103/05006; C12Y 201/01011; C12Y 606/01001

(57) ABSTRACT

Disclosed are modified strains of Chlorella vulgaris having a very low chlorophyll content. Also disclosed is a method for producing them. The method involves performing mutagenesis of a parental strain of Chlorella vulgaris. Furthermore, disclosed is a composition comprising algae biomass derived from the modified strains of Chlorella vulgaris and their use in food and/or cosmetics amongst other applications.

### INCORPORATION BY REFERENCE

The sequence listing in XML format is incorporated herein by reference in its
a. Name of File: ALGEN003US1_Sequence Listing_ST25
b. Date of Creation: 27 Mar. 2024
c. Size of File: 33,488 Bytes

Applicant submits that the “Sequence Listing” includes no new matter.

### TECHNICAL FIELD

The present disclosure relates generally to algae or microalgae and more specifically to modified strains of Chlorella vulgaris having a very low chlorophyll content. The present disclosure relates to modified strains of Chlorella vulgaris having a chlorophyll content lower than the chlorophyll content of wild-type strains of Chlorella vulgaris grown under the same conditions. Furthermore, the present disclosure also relates to methods of producing modified strains of Chlorella vulgaris having chlorophyll content lower than the chlorophyll content of wild-type strains of Chlorella vulgaris grown under the same conditions. Moreover, the present disclosure relates to compositions comprising algae biomass derived from the aforementioned modified strains of Chlorella vulgaris or obtained by performing the aforementioned methods. The present disclosure also relates to microalgae products comprising homogenates of microalgae biomass derived from the aforementioned modified strains of Chlorella vulgaris or obtained by performing the aforementioned methods.

### BACKGROUND

With current and projected increases in global human population there is an ever-increasing need to meet the nutritional requirements of all the individuals. Furthermore, in order to meet such nutritional requirements, acres of land are utilized around the world to grow crops and/or for development of plant-based food sources. In addition to the plant-based food sources, animal-based food sources such as poultry, cattle and seafood are also depended upon as a primary food source throughout the world and vast areas of land, food and water resources are required for the rearing of animals for human consumption. Dedicating such enormous amounts of land, food and/or water for the rearing of animals for consumption has been deemed to be problematic, owing to the growing need of the resources for livelihood of the growing human population. Furthermore, it has been observed that in the course of supplying food for humans, animals are slaughtered in large numbers, thereby impacting a balanced ecosystem (for example, leading to an increase in emission of greenhouse gases, a reduction in animal population and so forth). Therefore, there is an increased demand for additional food sources, able to produce nutritious and palatable food ingredients in a cost-effective and easy way.

Recently, fungi, algae, phytoplankton, zooplankton and so forth have been identified as potential sources of food, biofuels, cosmetic, pharmaceutical or nutraceutical ingredients, for chemical applications and so forth. For example, algae are simple, non-flowering plants requiring only water, sunlight and a few nutrients for their cultivation. Algae may range from microscopic algae (or “microalgae”, such as phytoplankton) to multicellular algae (or “macroalgae”, such as seaweed). Macroalgae, such as seaweed and kelp have been traditionally used as a food source for both human and animal consumption.

Besides macroalgae, microalgae have also been identified as a potential source of essential nutrients that provide several other benefits. The green microalgae, Chlorella vulgaris, has been identified as a superfood and is exempt from EU Novel Food Regulation (EU) 2015/2283-being “on the market as a food or food ingredient and consumed to a significant degree [within the EU] before 15 May 1997” (safe to eat for both humans and animals both as a whole food and as an ingredient) as well as being present on the CIRS China List of cosmetic ingredients both as whole cell and as extract as well as being included on the European Cosmetics Ingredients list.

Despite the advantages offered, the use of Chlorella vulgaris has been limited at least in part for certain market applications, including acceptance as a conventional food source and widespread use as a cosmetics and/or personal care ingredient. The limited use of Chlorella vulgaris is largely due to the dark-green colour, along with undesirable aroma and flavour that are often associated with the normal levels of chlorophyll in the wild-type Chlorella vulgaris), usually between 1-2% of the dry cell weight of this organism, Chlorella vulgaris.

In order to overcome these problems, to promote their use in food or as food ingredients, Chlorella vulgaris biomass is either used for specific products and markets where acceptance would be more expected in spite of the less-appealing colour, appearance and/or taste and smell, or used at very low incorporation rate, or often mixed with other components (food or food ingredients) with a different colour, stronger aroma and/or flavour or omitted from certain products/markets altogether. However, the latter techniques may still fail to overcome the undesirable colour, aroma and/or flavour associated with Chlorella vulgaris. Consequently, these microalgae do not have the most desirable properties to be used as food, cosmetic and/or personal care ingredients.

There exists a need to overcome the aforementioned drawbacks associated with Chlorella vulgaris microalgae and their use for human consumption and other market needs.

### SUMMARY

The present invention overcomes the highlighted drawbacks and provides modified strains of Chlorella vulgaris having a very low chlorophyll content.

The new strains of Chlorella vulgaris have extremely low chlorophyll content. Moreover, the new strains overcome the problem of undesirable organoleptic properties associated with wild-type microalgae in general and Chlorella vulgaris in particular and are safe and suitable for human consumption and to be used as food, cosmetic and personal care ingredients amongst other applications. In addition, the strains of the invention not only have an extremely low chlorophyll content but are competent heterotrophs (i.e. they are cultivable solely on an organic carbon source, in the absence of light). Furthermore, the new strains of the invention have stable pigmented phenotypes (i.e. the pigment content and consequently, the colour, of each strain is a property of its genotype).

The present invention also provides a modified strain of Chlorella vulgaris having a chlorophyll content lower than the chlorophyll content of a wild-type strain of Chlorella vulgaris from which it is derived, when grown under the same conditions.

The present disclosure also seeks to provide a method of producing a modified strain of Chlorella vulgaris having a very low chlorophyll content.

The present disclosure also seeks to provide a method of producing a modified strain of Chlorella vulgaris having a chlorophyll content lower than the chlorophyll content of a wild-type strain of Chlorella vulgaris from which it is derived, when grown under the same conditions.

According to one aspect, an embodiment of the present disclosure provides a modified strain of Chlorella vulgaris having a chlorophyll content in a range of 0.001 to 0.5 mg/g dry cell weight (DCW).

According to another aspect, an embodiment of the present disclosure provides a modified strain of Chlorella vulgaris having a chlorophyll content lower than the chlorophyll content of a wild-type strain of Chlorella vulgaris from which it is derived, when grown under the same conditions. The chlorophyll content of the modified strain is 0.5 to 0.001 mg/g DCW.

The modified strain of Chlorella vulgaris has substantially reduced chlorophyll content and consequently, is not associated with an unpleasant colour, smell (fragrance) and/or taste (flavour) associated with chlorophyll, making it suitable for use in food or as an ingredient in food products, nutraceutical formulations, cosmetics, personal care products and so forth. Notably, the modified strain of Chlorella vulgaris is genetically stable and can be grown in a broad range of conditions, ranging from optimal to stressful conditions, over time and not just limited to use of light (sunlight or artificial light).

The modified strain of Chlorella vulgaris of the invention is a heterotroph. Optionally, the invention provides a modified strain of Chlorella vulgaris having a chlorophyll content lower than the chlorophyll content of a wild-type strain of Chlorella vulgaris from which it is derived, when grown under the same conditions, preferably heterotrophic conditions, wherein the modified strain of Chlorella vulgaris has a chlorophyll content in a range of 0.001 to 0.5 mg/g DCW.

Optionally, the modified strain of Chlorella vulgaris has a chlorophyll content in a range of at least 90% to 99.9% lower than the chlorophyll content of the wild-type strain of Chlorella vulgaris grown under the same conditions. Preferably the modified strain of Chlorella vulgaris has a chlorophyll content in a range of at least 95% to 98% lower than the chlorophyll content of the wild-type strain of Chlorella vulgaris grown under the same conditions.

Optionally, the modified strain of Chlorella vulgaris has a chlorophyll content below 10%, more optionally below 5%, yet more optionally below 2%, yet more optionally below 1%, yet more optionally below 0.5%, and yet more optionally up to 0.1% of the chlorophyll content of the wild-type strain of Chlorella vulgaris grown under the same conditions.

In another aspect, the modified strain of Chlorella vulgaris of the invention has a chlorophyll content of 0.50 to 0.25 mg/g DCW, preferably 0.25 to 0.10 mg/g DCW, and most preferably 0.1 to 0.001 mg/g DCW.

Optionally, the modified strain of Chlorella vulgaris has at least one of a white, cream, pale yellow, yellow, pale green, golden, caramel, orange, pink, red, brown or lime colour. Optionally the modified strain of Chlorella vulgaris has a pigment composition linked to at least one colour selected from white, cream, pale yellow, yellow, pale green, golden, caramel, orange, pink, red, brown or lime. Typically, each colour may be defined by the relative levels of key pigments.

Optionally, the modified strain of Chlorella vulgaris is obtained from a parental strain of Chlorella vulgaris by performing mutagenesis of the parental strain of Chlorella vulgaris. The parental strain may be a wild-type strain of Chlorella vulgaris or a variation of the wild-type strain of Chlorella vulgaris. The variation of the wild-type strain of Chlorella vulgaris is a non-recombinant genetic variant.

Optionally, the modified strain of Chlorella vulgaris is obtained from the wild-type strain of Chlorella vulgaris by performing in vivo mutagenesis of the wild-type strain of Chlorella vulgaris.

More optionally, the mutagen is chemical or physical. Preferably the mutagen is chemical. More preferably, the mutagen is an alkylating agent. The classes of alkylating agents functioning as mutagens include but are not limited to sulphur mustards, nitrogen mustards, epoxides, ethylene imines, alkyl alkanesulphonates, dialkyl sulphates, beta-lactones, diazo compounds and nitroso compounds. Examples of such alkylating agents from each of these respective classes include: mustard gas, nitrogen mustard (HN2), ethylene oxide (EO), diepoxybutane (DEB), ethyleneimine (EI), triethylenemelamine (TEM), ethyl methanesulphonate (EMS) and methyl methansulphonate (MMS), diethylsulphate (DES), beta-propiolactone, diazomethane, N-Nitroso-N-methylurea (NMU) and N-methyl-N′-nitro-N-nitrosoguanidine (NG or NTG or MNNG) (as described by Auerbach 1976; DOI: 10.1007/978-1-4899-3103-0_16). Generally, the alkylating agents transfer alkyl groups (such as methyl or ethyl group) to macromolecules (such as bases, or the backbone phosphate groups of the nucleic acids) under physiological conditions. Typically, the alkyl group acts on nucleophilic sites of the macromolecule, for example, nitrogen or oxygen nucleophiles in DNA (Gates 2009; DOI: 10.1021/tx900242k). Such transfers result in alkylation of bases (for example guanine) and subsequent mispairing of said base during DNA replication (with for example, thymine instead of cytosine). It will be appreciated that the alkylating agents function similar to EMS in producing mutations in the genetic makeup of the organism exposed thereto. Repeated replication of such mispaired DNA can result in a transition mutation, wherein original G:C base pairs change to A:T base pairs, thereby changing the genetic makeup of the organism. In such case, the replication of such mutated DNA may create heritable missense mutations or nonsense mutations within coding sequences or impacting gene expression or gene function by compromising regulatory sequence functionality including RNA splice-site mutations. Beneficially, the in vivo application of alkylating agents as mutagenic chemicals for plant breeding, for human consumption, is not considered to produce Genetically Modified Organism (GMOs) as defined by the current EU legislation (2001/18/EC; Annex 1B, further clarified by the Court of Justice of the European Union Judgment in Case C-528/16 of 25 Jul. 2018), and is therefore acceptable for further applications in various industries, such as food, health, biotechnology and biofuels. This type of chemical mutagenesis is identical to that used in mutation breeding of food plants—a technique in common use for the production of new plant varieties since the early 1960's (Shu et al 2011; ISBN: 978-92-5-105000-0, Oladosu et al 2015; DOI: 10.1080/13102818.2015.1087333). Consequently, chemical mutagenesis using alkylating agents such as EMS is a technique for in vivo mutagenesis has conventionally been used in a number of applications and has a long safety record.

The in vivo method of mutagenesis here employed does not include the use of recombinant genetic techniques or employ the use of heterologous DNA—i.e. it is the conventionally established or “classical” mutagenesis technique previously described; in which all mutations are isolated within the target (“parent”) organism.

The technique of in vivo mutagenesis is clearly differentiated from in vitro mutagenesis, because the latter is a recombinant technique in which genes are cloned and then modified outside of their genetic context (i.e. outside of their parent organism). The differences between in vivo and in vitro mutagenesis have long been defined in the scientific literature, for example Botstein and Shortle (1985), DOI: 10.1126/science.2994214.

Optionally, a variation of the wild-type strain of Chlorella vulgaris, is treated with a mutagen and colour variants are then selected visually based on appearance after growth on heterotrophic growth medium.

Optionally, the wild-type strain of Chlorella vulgaris or a variation of the wild-type strain, is cultivated in the presence of a mutagen and colour variants are then selected visually based on appearance after growth on solid medium.

Preferably, the mutagenesis is performed by exposure of the strain of Chlorella vulgaris to a mutagenic chemical. More optionally, the mutagenic chemical is ethyl methanesulphonate.

Optionally, the quantity of the mutagenic chemical is in a range of 0.1 to 2.0 M. Preferably, the quantity of the mutagenic chemical is in a range of 0.1 to 1.0 M.

Optionally, the mutagenesis is performed by exposure of the wild-type strain of Chlorella vulgaris to a non-lethal quantity of a mutagenic chemical. More optionally, the mutagenic chemical is ethyl methanesulphonate.

Optionally, the non-lethal quantity of the mutagenic chemical is in a range of 0.1 to 2.0 M. Preferably, the quantity of the mutagenic chemical is in a range of 0.1 to 1.0 M.

Optionally, the modified strain of Chlorella vulgaris comprises a mutation at one or more positions in a nucleic acid sequence, and wherein each mutation is one of: a substitution, an insertion, or a deletion relative to a native strain of Chlorella vulgaris, and wherein the modified strain has a chlorophyll content in a range of 0.001 to 0.5 mg/g dry cell weight, preferably 0.25 to 0.50 mg/g dry cell weight, 0.10 to 0.25 mg/g dry cell weight or 0.001 to 0.1 mg/g dry cell weight.

Optionally, the mutation is a transition mutation.

Optionally, the mutation is in any of a coding region or a non-coding region of the nucleic acid sequence, and wherein mutation in the coding region results in any of: a neutral gene expression, an altered gene expression and/or a modified amino acid sequence.

Optionally, the one or more mutated gene is associated with at least 90% reduced gene expression relative to the native strain of Chlorella vulgaris, wherein the reduced gene expression results in the chlorophyll content in a range of 0.001 to 0.5 mg/g dry cell weight in the modified strain of Chlorella vulgaris.

Optionally, the mutation results in a one or more mutated genes, and wherein the one or more mutated genes is associated with a metabolic change in: a tetrapyrrole biosynthesis pathway, a chlorophyll biosynthesis pathway, a carotenoid biosynthesis pathway, or other pathways.

Optionally, the nucleic acid sequence has one or more mutations encoding for amino acid sequences having mutations in magnesium chelatase subunit I (as set forth in SEQ ID NO: 1), O-methyltransferase (as set forth in SEQ ID NO: 2), magnesium-protoporphyrin O-methyltransferase (as set forth in SEQ ID NO: 3), 15-cis-phytoene desaturase (as set forth in SEQ ID NOs: 4 and 5), zeta-carotene desaturase (as set forth in SEQ ID NOs: 6 and 7).

Optionally, mutated genes within the same strain have mutations encoding for amino acid sequences having mutations in both magnesium chelatase subunit I (as set forth in SEQ ID NO: 1) and ferrochelatase-1 (as set forth in SEQ ID NO: 8).

Optionally, the modified amino acid sequence comprises codon replacement of one codon for another codon, and wherein the modified codon codes for any of: a chemically similar amino acid, a different amino acid, or a stop codon.

Optionally, the reduced chlorophyll content is associated with at least one of chlorophyll a (α-chlorophyll) and/or chlorophyll b (β-chlorophyll) and collectively, the reduction in chlorophyll content is in a range of at least 90% to 99.9% as compared to the wild-type strain of Chlorella vulgaris grown under the same conditions. Preferably the reduction in chlorophyll content is in a range of at least 95% to 98%. Preferably the chlorophyll content is 0.5 to 0.25 mg/g DCW, more preferably 0.25 to 0.1 mg/g DCW, and most preferably 0.1 to 0.001 mg/g DCW.

Optionally, the modified strain of Chlorella vulgaris is obtained after cultivation under heterotrophic growth mode.

Optionally, the modified strain of Chlorella vulgaris is obtained after cultivation:
at a specific temperature in a range of 20 to 35° C.,
for a predefined period of time, typically for a period of 1 to 5 weeks,
without presence of light, and
in the presence of an organic carbon energy source.

More optionally, the specific temperature is in a range of 25 to 30° C., preferably in a range of 25 to 28° C., most preferably above 28° C.

Optionally, the predefined period of time is in a range of 1 to 3 weeks.

Optionally, the organic carbon energy source is glucose or acetate.

Optionally, the modified strain of Chlorella vulgaris is cultivated in the dark.

Optionally, the modified strain of Chlorella vulgaris has a lutein content lower than the lutein content of the wild-type strain of Chlorella vulgaris, normally in a range of 3 to 10 mg/g dry cell weight (DCW) when grown heterotrophically.

Optionally, the modified strain of Chlorella vulgaris has a lutein content below 9 mg/g DCW, more optionally below 8 mg/g DCW, yet more optionally below 7 mg/g DCW, yet more optionally still below 6 mg/g DCW, yet more optionally still below 5 mg/g DCW, yet more optionally below 4 mg/g DCW, yet more optionally still below 3 mg/g DCW, yet more optionally still below 2 mg/g DCW, yet more optionally still below 1 mg/g DCW, and yet more optionally up to 0.1 mg/g DCW.

Optionally, the modified strain of Chlorella vulgaris has a minimum protein content of at least 20%, or optionally 25%, or optionally 30%, or optionally 35%, or optionally 40%, or optionally 45% and still more optionally 50% w/w.

The modified strain of Chlorella vulgaris of the invention is genetically stable with respect to the observed colour phenotype.

Optionally, a modified strain of Chlorella vulgaris is derived from any one of: the wild-type strain of Chlorella vulgaris or a variation of the wild-type strain of Chlorella vulgaris, the modified strain having a chlorophyll content lower than a chlorophyll content of any one of: the wild-type strain of Chlorella vulgaris or the variation of the wild-type strain of Chlorella vulgaris from which it is derived, when grown under the same conditions, preferably heterotrophic conditions, and wherein the chlorophyll content of the modified strain is 0.001 to 0.5 mg/g DCW.

According to another aspect, an embodiment of the present disclosure provides a method of producing a modified strain of Chlorella vulgaris having a chlorophyll content in the range of 0.5 to 0.001 mg/g DCW, characterised in that the method comprises:
a) obtaining the parental strain of Chlorella vulgaris and defining the strain genetically as Chlorella vulgaris using molecular methods of taxonomic identification, such as PCR amplification, sequencing and alignment of 18S ribosomal RNA gene sequences, sequencing and alignment of the internally transcribed spacer (ITS) regions between the 18S rRNA, 5.8S rRNA and the 28S rRNA gene sequences, whole genome sequences or markers;
b) performing mutagenesis of the parental strain of Chlorella vulgaris;
c) cultivating the mutated strains of Chlorella vulgaris at a specific temperature, for a predefined period of time, in the dark and in the presence of an organic carbon energy source; and
d) identifying colonies of the mutated strain of Chlorella vulgaris having a phenotype different from the parental strain of Chlorella vulgaris as the modified strain of Chlorella vulgaris.

Another embodiment of the present disclosure provides a method of producing a modified strain of Chlorella vulgaris having a chlorophyll content lower than the chlorophyll content of a wild-type strain of Chlorella vulgaris grown under the same conditions, characterised in that the method comprises:
a) obtaining the wild-type strain of Chlorella vulgaris and defining the strain genetically as Chlorella vulgaris using molecular methods of taxonomic identification such as PCR amplification, sequencing and alignment of 18S ribosomal RNA gene sequences, sequencing and alignment of the internally transcribed spacer (ITS) regions between the 18S rRNA, 5.8S rRNA and the 28S rRNA gene sequences, whole genome sequences or markers;
b) performing mutagenesis of the wild-type strain of Chlorella vulgaris,
c) cultivating the mutated strains of Chlorella vulgaris at a specific temperature, for a predefined period of time, in the dark and in the presence of an organic carbon energy source; and
d) identifying colonies of the mutated strain of Chlorella vulgaris having a phenotype different from the wild-type strain of Chlorella vulgaris as the modified strain of Chlorella vulgaris.

Optionally, the method of producing a modified strain of Chlorella vulgaris having a chlorophyll content in the range of 0.001 to 0.5 mg/g DCW comprises:
a) obtaining the wild-type strain of Chlorella vulgaris and defining the strain genetically as Chlorella vulgaris using molecular methods of taxonomic identification such as PCR amplification, sequencing and alignment of 18S ribosomal RNA gene sequences, sequencing and alignment of the internally transcribed spacer (ITS) regions between the 18S rRNA, 5.8S rRNA and the 28S rRNA gene sequences, whole genome sequences or markers;
b) performing mutagenesis of the wild-type strain of Chlorella vulgaris;
c) cultivating the mutated strains of Chlorella vulgaris at a specific temperature, for a predefined period of time, in the dark, and in the presence of an organic carbon energy source; and
d) identifying colonies of the mutated strain of Chlorella vulgaris having a phenotype different from the wild-type strain of Chlorella vulgaris as the modified strain of Chlorella vulgaris.

Optionally, the modified strain of Chlorella vulgaris is a heterotroph.

Optionally, the method comprises obtaining the modified strain of Chlorella vulgaris from a parental strain of Chlorella vulgaris by performing mutagenesis of the parental strain of Chlorella vulgaris, wherein the parental strain is any one of a wild-type strain of Chlorella vulgaris or a variation (i.e. a genetic variant or genetic mutant) of the wild-type strain of Chlorella vulgaris. Optionally, the mutagenesis is performed by exposure of the wild-type strain of Chlorella vulgaris to a non-lethal quantity of a mutagenic chemical for a specific time. Optionally, the specific time for treatment with the mutagenic chemical is 1 to 120 minutes. More optionally, the mutagenic chemical is ethyl methanesulphonate.

Optionally, the method comprises using a non-lethal quantity of the mutagenic chemical is in a range of 0.1 to 2.0M. Preferably, the quantity of the mutagenic chemical is in a range of 0.1 to 1.0 M.

Optionally, the method comprises performing mutagenesis by exposing the wild-type strain of Chlorella vulgaris to a physical mutagen, wherein the physical mutagen comprises at least one of: UV light, gamma rays, X-rays.

Optionally, the method comprises identifying a modified strain of Chlorella vulgaris by sorting the cells by flow cytometry.

Optionally, the method comprises selecting a modified strain of Chlorella vulgaris based on a desirable protein content, wherein the desirable protein content is based upon a relative signal obtained on cell sorting by flow cytometry.

Optionally, the method comprises selecting a modified strain of Chlorella vulgaris based on a phenotype observed under manufacturing conditions, including but not limited to: colour, pigment composition, colour or pigment stability following heat treatment, macronutrient composition, micronutrient composition, growth rate, productivity and the like.

Advantageously, preferred strains may also be homogenised in a composition comprising a food, a beverage, a personal care product, a cosmetic and the like, without exhibiting separation or visible particles following the production process.

Optionally, the method further comprises selecting healthy colonies (or filtering out unhealthy colonies) of the modified strain of Chlorella vulgaris, preferably by cultivation under non-permissive or stressful conditions.

Optionally, the method further comprises performing steps (b) to (d) repeatedly for selecting healthy colonies of the modified strain of Chlorella vulgaris based on desired traits, wherein the desired traits comprise a colour, a pigment content, a protein content, improved tolerance to process conditions or macromolecular composition.

Optionally, the method comprises selecting the healthy colonies of the modified strain of Chlorella vulgaris by flow cytometry.

Optionally, the method comprises cultivating the mutated strains of Chlorella vulgaris at specific temperature is in a range of 20 to 35° C., preferably above 28° C., at, the predefined period of time in a range of 1 to 3 weeks, preferably in dark conditions, and in the presence of the organic carbon energy source such as glucose or acetate.

Optionally, the phenotype in d) is colour or another scorable visual phenotype. Optionally, the phenotype is at least one of: a colour, a smell (fragrance), a taste (flavour) or a texture.

Optionally, the method comprises producing modified strain of Chlorella vulgaris having one of: white, cream, pale yellow, yellow, pale green, golden, caramel, orange, pink, red, brown or lime colour. Typically, the colour may be determined by visual inspection of the strains. Other methods may also be used to determine and measure the colour of the strains.

Optionally, the method comprises cultivating the modified strain of Chlorella vulgaris using one or more of: a liquid or solid growth medium, a mixotrophic growth medium or a heterotrophic growth medium.

According to a further aspect, an embodiment of the present disclosure provides a composition comprising an algae biomass derived from a modified strain of Chlorella vulgaris, the modified strain having a chlorophyll content in a range of 0.001 to 0.5 mg/g DCW. Typically, the modified strain of Chlorella vulgaris is a heterotroph.

Disclosed herein is also a modified strain of Chlorella vulgaris which is a mixotroph and is obtained by the methods described herein.

Optionally, the composition comprising an algae biomass derived from a modified strain of Chlorella vulgaris having a chlorophyll content lower than the chlorophyll content of a wild-type strain of Chlorella vulgaris grown under the same conditions, or obtained by performing a method of producing a modified strain of Chlorella vulgaris having a chlorophyll content lower than the chlorophyll content of a wild-type strain of Chlorella vulgaris grown under the same conditions.

In another aspect, a composition of the invention comprises an algae biomass derived from a modified strain of Chlorella vulgaris having a chlorophyll content of 0.50 to 0.25 mg/g DCW, preferably 0.25 to 0.10 mg/g DCW, and most preferably 0.1 to 0.001 mg/g DCW.

Optionally, the composition is a food or food ingredient.

Optionally, the composition is a cosmetic or cosmetic ingredient.

Optionally, the composition is employed in at least one of: human foods, human nutraceutical preparations or formulations, animal feeds, pharmaceutical compositions including vaccines, cosmetics, personal care compositions, personal care devices or textiles, dyes or inks.

According to another aspect, an embodiment of the present disclosure provides a method of using the aforesaid composition as an ingredient in at least one of: human foods, human nutraceutical preparations or formulations, animal feeds, pharmaceutical compositions including vaccines, cosmetics, personal care compositions, personal care devices or textiles, dyes or inks.

Another embodiment of the present disclosure provides a microalgae product or flour comprising a homogenate of microalgae biomass derived from a modified strain of Chlorella vulgaris, the modified strain having a chlorophyll content of 0.5 to 0.001 mg/g DCW.

Optionally, a microalgae product or flour comprising a homogenate of microalgae biomass derived from a modified strain of Chlorella vulgaris having a chlorophyll content lower than the chlorophyll content of a wild-type strain of Chlorella vulgaris grown under the same conditions, or obtained by performing a method of producing a modified strain of Chlorella vulgaris having a chlorophyll content lower than the chlorophyll content of a wild-type strain of Chlorella vulgaris grown under the same conditions.

Another embodiment of the present disclosure provides an algae biomass derived from a modified strain of Chlorella vulgaris having a chlorophyll content of 0.5 to 0.001 mg/g DCW.

Optionally, an algae biomass is derived from a modified strain of Chlorella vulgaris having a chlorophyll content lower than the chlorophyll content of a wild-type strain of Chlorella vulgaris grown under the same conditions, or obtained by performing a method of producing a modified strain of Chlorella vulgaris having a chlorophyll content lower than the chlorophyll content of a wild-type strain of Chlorella vulgaris grown under the same conditions.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is an illustration of steps of a method of producing a modified strain of Chlorella vulgaris having a chlorophyll content lower than the chlorophyll content of a wild-type strain of Chlorella vulgaris grown under the same conditions, in accordance with an embodiment of the present disclosure;

FIG. 2 show a thin-layer chromatography analysis of pigments in a panel of wild-type and colour mutants, in accordance with an embodiment of the present disclosure;

FIG. 3 show a comparison of wild-type (WT), yellow (YC01) and lime (YC02) Chlorella vulgaris strains cultivated in liquid culture, in accordance with an embodiment of the present disclosure;

FIG. 4 show a comparison of various modified strains Chlorella vulgaris, i.e. YC06, YC03, YC10, YC18, YC24, YC14 and YC20, cultivated in liquid culture, in accordance with an embodiment of the present disclosure;

FIGS. 5-8 show chlorophyll content in chlorophyll deficient colour variants as compared to the chlorophyll content produced in wild-type cells for the parental strain (4TC3/16) and a comparative, well characterised culture collection strain of Chlorella vulgaris (CCAP 211/11b) cultivated under heterotrophic conditions using acetate or glucose as the primary carbon source, in accordance with various embodiments of the present disclosure;

FIGS. 9 and 10 show chlorophyll content in chlorophyll deficient colour variants represented as a percentage of chlorophyll produced in wild-type cells cultivated under heterotrophic conditions using acetate or glucose as the primary carbon source, in accordance with various embodiments of the present disclosure;

FIGS. 11-14 show tables of chlorophyll content of Chlorella vulgaris colour variants and wild-type cells, grown under the same conditions, represented as the average of three measurements and expressed in micrograms, in accordance with various embodiments of the present disclosure;

FIG. 15 shows an example of the use of flow cytometry to identify subpopulations of cells (white YC20 strain and yellow YC03 strain) based on pigment composition. Black contours show a representative population from the white YC20 strain. Grey contours show a representative cell population from the yellow YC03 strain;

FIG. 16 shows a table of chlorophyll-deficient EMS mutants of Chlorella vulgaris 4TC3/16 (wild-type) strain, in accordance with an embodiment of the present disclosure;

FIG. 17 shows an overview of chlorophyll biosynthesis in Chlorella vulgaris, in accordance with an embodiment of the present disclosure;

FIG. 18 shows an overview of carotenoid biosynthesis in Chlorella vulgaris, in accordance with an embodiment of the present disclosure;

FIG. 19 shows an overview of chlorophyll biosynthesis in Chlorella vulgaris red variant (Red8), in accordance with an embodiment of the present disclosure;

FIG. 20 shows Chlorella vulgaris red variant (Red8) cultivated in liquid culture, in accordance with an embodiment of the present disclosure; and

FIG. 21 shows a thin-layer chromatography analysis of pigments in Chlorella vulgaris red variant (Red8), in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item.

When a number is non-underlined and accompanied by an associated arrow, the non-underlined number is used to identify a general item at which the arrow is pointing.

### DETAILED DESCRIPTION OF EMBODIMENTS

In overview, embodiments of the present disclosure are concerned with a modified strain of Chlorella vulgaris having a chlorophyll content lower than the chlorophyll content of a wild-type strain of Chlorella vulgaris grown under the same conditions. Furthermore, embodiments of the present disclosure are concerned with a method of producing the modified strain of Chlorella vulgaris.

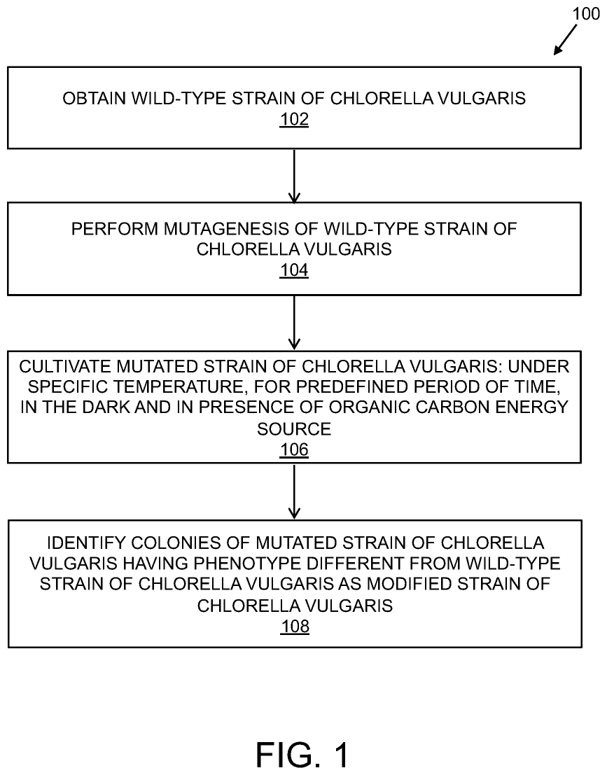

FIG. 1 shows steps of a method 100 of producing a modified strain of Chlorella vulgaris having a chlorophyll content lower than the chlorophyll content of a wild-type strain of Chlorella vulgaris grown under the same conditions, in accordance with an embodiment of the present disclosure. Throughout the present disclosure, the term “Chlorella vulgaris” as used herein, refers to a specifically identified (based on partial or whole genome sequence analysis) unicellular green algal species. Algae are photosynthetic organisms that grow in diverse habitats ranging from regions of varying hardness, humidity, salinity, light-access, and temperature, such as land, rivers, ponds, lakes, sea, brackish water, wastewater and the like. The species, Chlorella vulgaris, is a single-cell aquatic plant falling under the phylum/division “Chlorophyta” within the plant taxonomic kingdom (the full taxonomic assignment being: Biota Plantae (Kingdom) Viridiplantae (Subkingdom) Chlorophyta (Phylum (Division)) Chlorophytina (Subphylum (Subdivision)) Trebouxiophyceae (Class) Chlorellales (Order) Chlorellaceae (Family) Chlorella (Genus) Chlorella vulgaris (Species)). Chlorella vulgaris is a fast-growing microalga (or a microscopic single-celled organism). Chlorella vulgaris can also grow in areas that are unsuitable for cultivating plants by traditional agricultural techniques and can be harvested daily, for example, to serve as a natural food source to meet the nutritional requirements of humans and/or animals.

Throughout the present disclosure, the term “chlorophyll” as used herein refers to a group of green pigments contained in cells of green plants. Chlorophyll is essential for photosynthesis and allows photosynthetic organisms to absorb energy from sunlight. Specifically, chlorophyll enables the photosynthetic organisms to absorb blue and red light from the visible region of the electromagnetic spectrum. However, green light from the visible region of the electromagnetic spectrum is comparatively poorly absorbed (and is therefore reflected), thus, imparting the green colour to the chlorophyll-containing tissues of most photosynthetic organisms. It will be appreciated that the chlorophyll content is associated with at least one of: chlorophyll a (α-chlorophyll or Chl-a) and/or chlorophyll b (β-chlorophyll or Chl-b). Specifically, chlorophyll a (or α-chlorophyll) is present in all vascular and non-vascular plants, while chlorophyll b (or β-chlorophyll) is present in algae and green plants. More specifically, chlorophyll a is a primary photosynthetic pigment, which participates directly in the light-driven reactions of photosynthesis, while chlorophyll b is an accessory pigment operable to collect energy primarily from blue wavelengths of sunlight and pass it on to chlorophyll a.

The chlorophyll content in the wild-type strain of Chlorella vulgaris usually ranges between 0.1 and 1.5% of dry weight of the organism. Moreover, the chlorophyll content is influenced strongly by cultivation conditions, in particular the absence or presence of light. In the dark, chlorophyll content is naturally suppressed to as low as 0.1% dry weight of the organism, while in the light, chlorophyll content can be as high as 2% dry weight of the organism.

The wild-type strain of Chlorella vulgaris usually comprises chlorophyll a (Chl-a) and chlorophyll b (Chl-b) distributed in a ratio of approximately 4:1.

Moreover, the wild-type strain of Chlorella vulgaris may be used to produce generations of Chlorella vulgaris. In such instance the wild-type strain of Chlorella vulgaris serves as a parental strain for a successive generation of Chlorella vulgaris. Moreover, in the context of a mutagenesis campaign, the parental strain may be the wild-type strain of Chlorella vulgaris (as mentioned hereinabove) or a variation (namely, a modified version) of the wild-type strain of Chlorella vulgaris. The term “parental strain” as used herein, therefore may also refer to a genetic variant or subtype of Chlorella vulgaris, preferably a previous generation. The parental strain is characterized by differing genetic makeup as compared to successive generations that are derived from the parental strain by mutagenesis. The parental strain of Chlorella vulgaris can be obtained from its usual dwelling sites such as land, rivers, ponds, lakes, brackish water, wastewater and the like, or from an artificial site, such as laboratories, culture collections and so forth. The term “variation of the wild-type strain” as used herein, refers to a modified version of a wild-type strain due to mutations in the wild-type strain. It will be appreciated that the variation of the wild-type strain comprises a genetic makeup different from that of its parental strain, i.e. the wild-type strain, and exhibits phenotypes different from the normal parental strain. The variation of the wild-type strain of Chlorella vulgaris is genetically defined as Chlorella vulgaris using the previously described methods (also described in detail below).

At a step 102, the wild-type strain of Chlorella vulgaris is obtained and the strain is genetically defined as Chlorella vulgaris. The term “wild-type strain” as used herein, refers to a typical form of an organism as it occurs in nature, having a set of genes and exhibiting corresponding phenotypes associated with the organism as it would grow in its natural environment. The wild-type strain of Chlorella vulgaris can be obtained from its usual dwelling sites such as land, rivers, ponds, lakes, brackish water, wastewater and the like. The naturally-occurring wild-type strain of Chlorella vulgaris grows autotrophically by performing photosynthesis. During the process of photosynthesis, the wild-type strain of Chlorella vulgaris utilizes sunlight, carbon dioxide, water and a few nutrients to produce a biomass of alga. However, the wild-type strain of Chlorella vulgaris can also be cultivated using heterotrophic and/or mixotrophic growth modes. Wild-type strains of Chlorella vulgaris are haploid in their normal growth phase, i.e. have only one copy of the genome, thereby making Chlorella vulgaris particularly amenable to a phenotypic trait improvement approach using genetics as, for some traits, a single genetic change could yield the desired phenotype. Furthermore, being haploid, these strains are likely to be genetically stable as there is essentially no capacity of the mutant strain to easily correct or revert to the wild-type state; moreover, there is no other genetic copy of the DNA that can act as a correction template to facilitate this process. The wild-type strains of Chlorella vulgaris are associated with a dark-green colour, a specific smell or fragrance (such as aquatic, fish-like, earthy or mouldy smell) and an unpleasant taste or flavour. The wild-type strain of Chlorella vulgaris has a chlorophyll content that contributes to its unappealing dark-green colour, unpleasant smell (fragrance) and taste (flavour). A reduction of wild-type chlorophyll content below 90% and more preferably below 99.9% of wild-type levels is associated with the loss of the unappealing dark-green colour, unpleasant smell and taste.

The obtained wild-type strain of Chlorella vulgaris is genetically defined as Chlorella vulgaris using one or more of: a PCR amplification, sequencing and alignment of the genetic material such as nuclear and/or chloroplast DNA and/or ribosomal RNA (such as 18S rRNA, 5.8S rRNA and 28S rRNA), sequencing and alignment of the internally transcribed spacer (ITS) regions between the 18S ribosomal RNA (rRNA), 5.8S rRNA and the 28S rRNA, whole genome sequencing, and other markers. Typically, sequence comparisons of the internally transcribed spacer (ITS) regions have been used extensively for intra and inter genus phylogenetic analysis of the Chlorellaceae (green algae) family (Huss et al. 1999; DOI: 10.1046/j.1529-8817.1999.3530587.x, Krienitz et al., 2015; DOI: 10.1016/j.tplants.2014.11.005, Darienko and Pröschold 2015; DOI: 10.1111/jpy.12279, and Heeg and Wolf 2015; DOI: 10.1016/j.plgene.2015.08.001). Moreover, other statistics and additional sequences derived from whole genome sequencing is another useful method for strain identification.

PCR amplification employs primers associated with appropriate regions of the genome of a specific organism. Subsequently, the derived nucleotide sequences are compared to the nucleotide sequences of defined Chlorella vulgaris, with sequence alignments exhibiting sequence identities of 99.8% or greater with the defined nucleotide sequences confirming strain identity as the species Chlorella vulgaris, for further use in the present invention. Specifically, regions of conserved genomic DNA and/or rRNA gene sequences, can be amplified and compared to the corresponding regions of those preferred species. More specifically, species of Chlorella vulgaris, that exhibit at least 99.5% or greater nucleotide identity to at least one or more of the sequences obtained from isolates formally identified as the species Chlorella vulgaris are selected for the present invention. Consequently, sequencing of the test sequences, relative to identified (reference) sequence, enable determination of percent nucleotide identity, using a sequence alignment (comparison) algorithm, such as Smith & Waterman and Needleman & Wunsch homology algorithm and/or Pearson & Lipman similarity method. The sequence comparison algorithm then calculates the percent sequence identity for each of the test sequence(s). In an example, the identification of Chlorella vulgaris using PCR amplification, sequencing and alignment of 18S ribosomal RNA gene sequences excludes other species of Chlorella, other than the actual Chlorella vulgaris, from being selected. For example, alignment of 18S rRNA gene sequences of the sample identifies Chlorella vulgaris with 99.9% identity to the whole (or partial) 18S rRNA gene sequences of the defined Chlorella vulgaris, while a sample of a related but distinct microalgal species, Chlorella sorokiniana, would typically be 99.2% identical or less to the whole (or partial) 18S rRNA gene sequence of the defined Chlorella vulgaris.

At a step 104, mutagenesis of the wild-type strain of Chlorella vulgaris is performed, wherein the mutagenesis is performed by exposure of the wild-type strain of Chlorella vulgaris to a non-lethal quantity of a mutagenic chemical for a specific time. The term “mutagenesis” as used herein, relates to a technique of inducing mutations in an organism via a spontaneous natural process or by artificially exposing the organism to mutagens using laboratory procedures. The wild-type strain of Chlorella vulgaris is subjected to mutagenesis in order to produce variant or mutated strains of Chlorella vulgaris exhibiting a different phenotype, such as colour, from that exhibited by the wild-type strain of Chlorella vulgaris. Typically, the mutagenesis of the wild-type strain of Chlorella vulgaris is performed by exposing the wild-type strain of Chlorella vulgaris to a mutagenic chemical. Optionally the mutagenic chemical is selected from N-methyl-N-nitrosourea (NMU), methyl methane sulfonate (MMS), N-methyl-N′-nitro-N-nitrosoguanidine (MNNG) and ethyl methanesulphonate (EMS) nitrous acid (NA), diepoxybutane (DEB), 1, 2, 7, 8-diepoxyoctane (DEO), 4-nitroquinoline 1-oxide (4-NQO), 2-methyloxy-6-chloro-9 (3-[ethyl2-chloroethyl]-aminopropylamino)-acridinedihydrochloride (ICR-170), 2-amino purine (2AP), and hydroxylamine (HA). Preferably, the mutagenic chemical is and alkylating agent. More preferably, the mutagenic chemical is ethyl methanesulphonate (EMS). EMS favours certain types of chromosomal mutations rather than a general spectrum of mutagenesis.

Optionally, the mutation is a transition mutation. The mutagenic chemicals such as EMS produce random mutations, such as nucleotide substitutions, transition mutations and the like, in the genetic makeup of the organism exposed thereto. Typically, transition mutations are a point mutations involving substitution of one base pair for another by replacement of a purine nucleotide by another purine (i.e. A to G or vice versa) or a pyrimidine nucleotide by another pyrimidine (i.e. C to T or vice versa), while still maintaining the purine:pyrimidine ratio of the gene sequence. The use of EMS may result in a mutated ethylguanine base in the DNA as a result of guanine alkylation. Repeated replication of such mutated DNA can result in a transition mutation, wherein original G:C base pairs are replaced with A:T base pairs, thereby changing the genetic makeup of the organism at those sites. In such case, the replication of such mutated DNA may create missense mutations or nonsense mutations within coding sequences or impacting gene expression or gene function by compromising regulatory sequence functionality including splice-site mutations or mutations in key promoter or other regulatory elements.

Optionally, the modified strain of Chlorella vulgaris comprises a mutation at one or more positions in a nucleic acid sequence or an amino acid sequence, and wherein each mutation is one of: a substitution, an insertion, or a deletion relative to a native strain of Chlorella vulgaris, and wherein the modified strain has a chlorophyll content in a range of 0.001 to 0.5 mg/g dry cell weight, preferably 0.25 to 0.50 mg/g dry cell weight, 0.10 to 0.25 mg/g dry cell weight or 0.001 to 0.1 mg/g dry cell weight. The mutagenic chemical, such as EMS, results in a transition mutation by substituting original G:C base pairs with A:T/U base pairs at one or more positions on the nucleic acid sequence, i.e. DNA or RNA gene sequences, respectively.

Optionally, the mutation is in any of a coding region or a non-coding region of the nucleic acid sequence, and wherein mutation in the coding region results in any of: a neutral gene expression, an altered gene expression and/or a modified amino acid sequence. The coding regions of the nucleic acid sequence refers to a portion of the nucleic acid sequence that specifically codes for a certain kind of protein, while the non-coding regions are the portions of the nucleic acid sequence which do not code for any protein. Mutations in coding regions typically have diverse effect on the phenotype of the organism or survival thereof. Alternatively, the mutations in coding regions may not result in a detectable phenotypic change. Specifically, a silent mutation in the coding regions does not result in a corresponding amino acid change correlated with the neutral gene expression. On the other hand, a missense mutation or nonsense mutation in the coding region results in the altered gene expression coding for a modified amino acid during translation or producing a shorter final protein due to a premature termination of translation. Typically, the nucleic acid sequences code for proteins as well as other functional elements such as RNA gene sequences (such as transfer RNAs (tRNAs), microRNAs (miRNAs), and so forth) and regulatory gene sequences. Typically, mutations in non-coding regions may also impact gene expression by altering the promoter or regulatory sequences, termination sequences, ribosome binding sites, mRNA splice sites and so on, resulting in a modified amino acid sequence.

Optionally, the mutation results in a one or more mutated genes, and wherein the one or more mutated genes is associated with a metabolic change in: a tetrapyrrole biosynthesis pathway, a chlorophyll biosynthesis pathway, a carotenoid biosynthesis pathway, or other pathways. The one or more mutated genes encode for proteins, enzymes, transcription factors, and so on. Mutations in genes result in an altered functioning of said proteins, enzymes and/or transcription factors. Typically, the mutated genes may be associated with one or more metabolic pathways, including, but not limited to, the tetrapyrrole biosynthesis pathway, the chlorophyll biosynthesis pathway, the carotenoid biosynthesis pathway. The mutation in genes associated with the aforesaid pathways results in reduction in pigmentation and contributes to the phenotypes observed in the mutated strains. Optionally, the one or more mutated gene is associated with at least 90% reduced gene expression relative to the native strain of Chlorella vulgaris, wherein the reduced gene expression result in the chlorophyll content in a range of 0.001 to 0.5 mg/g dry cell weight in the modified strain of Chlorella vulgaris. The at least 90% reduction in gene expression is correlated with reduction in pigmentation in the mutated strains as compared to the native strains grown under same conditions.

Optionally, the combination of one or more mutated genes is correlated with a reduction in tetrapyrrole biosynthesis which results in a red phenotype, caused by corresponding accumulation of protoporphyrin IX, the precursor of both chlorophyll and heme proteins.

Optionally, the nucleic acid sequence has one or more mutations encoding for amino acid sequences having mutations in magnesium chelatase subunit I (as set forth in SEQ. ID NO: 1), O-methyltransferase (as set forth in SEQ. ID NO: 2), magnesium-protoporphyrin O-methyltransferase (as set forth in SEQ. ID NO: 3), 15-cis-phytoene desaturase (as set forth in SEQ. ID NOs: 4 and 5), zeta-carotene desaturase (as set forth in SEQ. ID NOs: 6 and 7). Optionally, the nucleic acid sequence has one or more further mutations encoding for amino acid sequences having mutations in ferrochelatase-1 (as set forth in SEQ. ID NO: 8); in this case, red strains of CV are obtained. The one or more mutated genes translate into any of magnesium chelatase subunit I, O-methyltransferase, magnesium-protoporphyrin O-methyltransferase, 15-cis-phytoene desaturase, zeta-carotene desaturase or ferrochelatase-1 enzymes. The said enzymes play a vital role in the chlorophyll biosynthesis and/or carotenoid biosynthesis and/or tetrapyrrole biosynthesis. Mutations in the genes encoding for said enzymes result in altered activity thereof, and thus a change in observed phenotypes in the organisms. Optionally, the modified amino acid sequence comprises codon replacement of one codon for another codon, and wherein the modified codon codes for any of: a chemically similar amino acid, a different amino acid, or a stop codon. Codons are a separate group of nucleotides that define specific amino acids. Typically, at least 3 nucleotides, read sequentially to form a codon, designate a specific amino acid. A modification in a nucleotide sequence results in a modified codon that codes for a specific amino acid. The degeneracy in the genetic code assigns one codon to code for one or more of the twenty fundamental amino acids (Alanine (Ala, A), Arginine (Arg, R), Asparagine (Asn, N), Aspartic acid (Asp, D), Cysteine (Cys, C), Glutamic Acid (Glu, E), Glutamine (Gln, Q), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y) and Valine (Val, V)). The modified codons may code for chemically similar amino acids (for example, the GA* block codes for Asp and Glu, both negatively charged amino acids) or chemically different amino acids (for example, UG* block codes for Cys, Trp and a stop codon) or stop codons (for example UAA, UAG and UGA). Thus, modified amino acid may or may not result in a detectable phenotypic change due to the degeneracy of the genetic code.

It will be appreciated that altered gene expression and modified amino acid sequence may have a positive effect or a negative effect on the overall health of the organism exposed to mutations. The mutation in gene sequence of the modified strain results in an altered phenotype as compared to the native strain such as a parental strain, a wild-type strain, or a defined Chlorella vulgaris strains (by conventional methods of sequencing as previously described). For example, modified strain of Chlorella vulgaris exhibits a different colour phenotype correlated with the low chlorophyll content in a range of 0.001 to 0.5 mg/g dry cell weight relative to the native strain of Chlorella vulgaris when exposed to the mutagenic chemical.

As mentioned, herein above, a non-lethal quantity of the mutagenic chemical is used for performing the mutagenesis. The term “non-lethal quantity” in the context of the invention means a quantity of a mutagenic chemical that does not kill 100% of the strain population (parental strain or strains). The exposure time to a given concentration of mutagen also influences its lethality. This process allows the selection of the variant strains that are able to survive the mutagenesis.

Optionally, the quantity of the mutagenic chemical is in a range of 0.1 to 2.0 M. Preferably, the quantity of the mutagenic chemical is in a range of 0.1 to 1.0 M. It will be appreciated that the quantity of the mutagenic chemical used for performing the mutagenesis, combined with the exposure time, can determine the amount of genetic change undergone by the organism. Furthermore, heavily mutagenized cells of the organism accumulate multiple mutations of genetic material, often resulting in deleterious alterations in the organism. This is of particular importance in a haploid organism, where only a single copy of each coding gene is present. It is common that multiple mutations occur within the genomes of mutagenized strains. Employing a high quantity of EMS, or a quantity of a mutagenic chemical over a period of time for performing the mutagenesis may result in point mutations which result in deleterious genetic changes in the mutated strain of Chlorella vulgaris as compared to the wild-type strain of Chlorella vulgaris or may result in death of the mutated strain of Chlorella vulgaris under the most severe scenarios. Therefore, using 0.1 to 1.0 M of the mutagenic chemical, such as EMS, over a determined exposure time enables to achieve desired phenotypes as balanced against excessive accumulation of undesirable traits that might reduce cell fitness, hamper growth, or result in death of the organism. Furthermore, by cultivation of the cells that have been exposed to mutagen at a slightly higher than ideal cultivation temperature a ‘stress’ filter has been effectively applied such that only the more robust strains where accumulated mutations have not produced a weakened or crippled organism can produce colonies on agar. By growth at this filtering temperature, fewer overall strains grow but those that do grow are more biologically and genetically fit with regard to growth/biomass production. Hence, those strains with reduced chlorophyll pigment that grow under these conditions and are scored based upon initial colour should also be expected to be more robust with regard to application within an ultimate scalable bioprocess.

Optionally, performing the mutagenesis of the wild-type strain of Chlorella vulgaris comprises exposing the wild-type strain of Chlorella vulgaris to a physical mutagen, wherein the physical mutagen comprises at least one of UV light, gamma rays or X-rays. These mutagens cause changes in the genomes of the wild-type strain of Chlorella vulgaris to result in mutated strains of Chlorella vulgaris. In such an instance, as an alternative to performing the mutagenesis of the wild-type strain of Chlorella vulgaris by exposure to the chemical mutagen, mutagenesis by exposure to physical mutagens can be performed to obtain the mutated strain of Chlorella vulgaris.

Optionally, the wild-type strain of Chlorella vulgaris is cultivated in the presence of a mutagen and colour variants are then selected visually based on appearance after growth on solid medium.

At a step 106, the mutated strain of Chlorella vulgaris is cultivated at a specific temperature, for a predefined period of time, without presence of light, and in the presence of an organic carbon energy source. Notably, algae such as Chlorella vulgaris can grow in conditions ranging from optimal to extreme and in varied habitats. Typically, the wild-type strain of Chlorella vulgaris is exposed to a chemical mutagen (such as EMS) or optionally to a physical mutagen, at constant temperature conditions. Typically, the wild-type strain of Chlorella vulgaris is exposed to the chemical mutagen or optionally, to a physical mutagen, for a predefined period of time. Such an exposure of the wild-type strain of Chlorella vulgaris at specific temperature conditions and for a predefined period of time enables derivation of modified (or mutated) strains of Chlorella vulgaris. Optionally, the specific temperature is in the range of 20 to 35° C., preferably, above 28° C., and the predefined period of time post-exposure is in a range of 1 to 5 weeks, preferably 1 to 3 weeks. In an example, the wild-type strain of Chlorella vulgaris is exposed to ethyl methanesulphonate at 25° C. for 2 hours.

Preferably, the mutated strain of Chlorella vulgaris is obtained from a wild-type strain of Chlorella vulgaris that can be cultivated without presence of light. The term “without presence of light” as used herein refers to cultivating in the dark or in the absence of light. In such case, as an example, the petri dishes containing the sample of Chlorella vulgaris may be wrapped individually in a substantially opaque sheet, such as a foil, and then the wrapped-up petri dishes may be placed inside a cardboard box in the incubator. Other suitable ways of cultivating in the dark or without the presence of light can be used.

Optionally, the mutated strain of Chlorella vulgaris of the invention is obtained from a wild-type strain of Chlorella vulgaris cultivated in the presence of low light. It will be appreciated that the mutated strain can grow in the presence of light so long as there is an exogenous carbon source such as acetate or glucose and that some mutations and resulting strains may be rendered hyper-sensitive to light such that cultivation above very low light levels, for instance 5 micromoles/m²/second light has an inhibitory or toxic effect on growth. Optionally, the characteristics of the light used (e.g. intensity of light, colour of light, and so forth) during mutagenesis, are defined. More optionally, the light intensity range and quality may be, i.e. white LED, white fluorescent, daylight fluorescent, red LED, or mix of white and red or other LED, with light intensity values ranging from 5 micromoles/m²/s to 300 micromoles/m²/s, most preferably 2 to 25 micromoles/m²/s of white LED light.

Optionally, the mutated strain of Chlorella vulgaris is obtained from a wild-type strain of Chlorella vulgaris cultivated using one or more of: a liquid or solid growth medium, including a fermentation medium containing an added carbon source such as glucose, or a mixotrophic growth medium containing acetate or a heterotrophic growth medium. In an example, the mutated strain of Chlorella vulgaris is obtained from a wild-type strain of Chlorella vulgaris cultivated using a solid medium. Such a solid medium can be a regular agar plate. In such an instance, cells of the mutated strain of Chlorella vulgaris are plated on agar plates at an appropriate cell plating density to achieve a dense colony distribution on the surface of the agar plates. It is important to note that addition of a simple carbon source such as, but not limited to, glucose (dextrose), acetate or other simple carbon compound allows the cells to grow in the dark under heterotrophic growth mode. The solid medium can be a high salt medium-glucose agar plate, wherein the high salt medium-glucose agar plate comprises: a growth medium such as High Salt Medium™ (HSM™), glucose (for example, 1% w/v) and agar.

In another example, the mutated strain of Chlorella vulgaris is cultivated using a liquid medium. Such a liquid medium can be at least one of TAP (Tris-Acetate-Phosphate), High Salt Medium™ (HSM™), glucose (for example, having consistency of 1% w/v) and so forth. The fermentation medium comprises a source of nitrogen (such as proteins or nitrate or, more usually, ammonium), minerals (including magnesium, phosphorus, potassium, sulphur, calcium, and iron), trace elements (zinc, cobalt, copper, boron, manganese, molybdenum), an optional pH buffer, a source of carbon and energy (such as glucose, acetate) and so forth. Optionally, the wild-type strain of Chlorella vulgaris is cultivated in a fermenter.

Preferably the mutated strain of Chlorella vulgaris is cultivated under heterotrophic growth mode. In other words, the mutated strain of Chlorella vulgaris is a heterotroph. For example, the mutated strain of Chlorella vulgaris is cultivated under heterotrophic growth mode without any presence of light (i.e. in the dark). In such an example, heterotrophic growth of the mutated strain of Chlorella vulgaris is achieved under suitable aseptic conditions. The heterotrophic growth can be carried out by growing the mutated strain of Chlorella vulgaris using a source of carbon and energy, such as glucose, without the presence of light. Alternatively, the mutated strain of Chlorella vulgaris is cultivated under mixotrophic growth mode with partial presence of light, such as by exposure of the mutated strain of Chlorella vulgaris to light for a limited time per day or at a minimally set light intensity. In such an example, the mixotrophic growth is performed by employing simultaneous use of different sources of energy for cultivating the mutated strain of Chlorella vulgaris. Furthermore, the Chlorella vulgaris uses different sources of energy, along with light, in different combinations for growth.

At a step 108, colonies of the mutated strain of Chlorella vulgaris having a phenotype different from the wild-type strain of Chlorella vulgaris are identified as the modified strain of Chlorella vulgaris. For example, when the mutated strain of Chlorella vulgaris is cultivated using agar plates, colonies of the mutated strain of Chlorella vulgaris on the agar plates that exhibit a different phenotype than the wild-type strain of Chlorella vulgaris are identified as the modified strain of Chlorella vulgaris. Optionally, the phenotype is at least one of: a colour or a smell (fragrance). Preferably, colour is used as the primary method to screen for the modified strain of Chlorella vulgaris from the wild-type strain of Chlorella vulgaris. In such an instance, the colonies of the mutated strain of Chlorella vulgaris are screened visually to identify the different phenotype than the phenotype of the wild-type strain of Chlorella vulgaris. Optionally, the colour of the modified strain of Chlorella vulgaris is one of: white, cream, pale yellow, yellow, pale green, golden, caramel, orange, pink, red, brown or lime colour, with the colour also being associated strongly with smell (fragrance) and taste (flavour). For example, the mutated strain of Chlorella vulgaris is incapable of producing, or has substantially reduced ability to produce, chlorophyll pigments (comprising chlorophyll a and/or chlorophyll b) and a variable but genetically determined ability to produce other pigments. Such colonies of the mutated strain of Chlorella vulgaris having the different colour, such as white, cream, pale yellow, yellow, pale green, golden, caramel, orange, pink, red, brown or lime colour, are identified as the modified strains of Chlorella vulgaris.

Optionally, the modified strain of Chlorella vulgaris has a chlorophyll content in a range of at least 90% to 99.9% lower than the chlorophyll content of the wild-type strain of Chlorella vulgaris grown under the same conditions. More optionally, the modified strain of Chlorella vulgaris has a chlorophyll content 90% lower, more optionally 95% lower, yet more optionally 98% lower and yet more optionally still 99.9% lower than the chlorophyll content of the wild-type strain of Chlorella vulgaris grown under the same conditions. Optionally, the modified strain of Chlorella vulgaris has a chlorophyll content below 10%, more optionally below 5%, yet more optionally below 2%, yet more optionally below 1%, and yet more optionally up to 0.1% of the chlorophyll content of the wild-type strain of Chlorella vulgaris grown under the same conditions. Preferably the modified strain of Chlorella vulgaris has a chlorophyll content of 0.50 to 0.25 mg/g DCW, more preferably 0.25 to 0.10 mg/g DCW, and most preferably 0.1 to 0.001 mg/g DCW. Notably, lower chlorophyll content of the modified strain of Chlorella vulgaris renders the modified strain of Chlorella vulgaris more commercially acceptable. For example, a modified strain of Chlorella vulgaris with chlorophyll content of 0.001 mg/g DCW will be more commercially acceptable in industries that require no colour in their final manufactured products, as compared to the modified strain of Chlorella vulgaris with chlorophyll content of 0.10 mg/g DCW.

It will be appreciated that the modified strain of Chlorella vulgaris comprises significantly reduced or negligible chlorophyll a and/or chlorophyll b content. Reduction or elimination of chlorophyll to the desired levels described herein does not have the effect to improve photosynthetic efficiency by reducing the light-harvesting chlorophyll antenna as has been described by others (e.g. Shin et al. 2016, DOI: 10.1007/s10811-016-0874-8; Shin et al. 2017, DOI: 10.1038/s41598-017-18221-0). Rather, the ability to grow photosynthetically is effectively abolished by the desired mutations described in this invention and our selection process is designed to specifically identify such variants. Such a reduced chlorophyll content of the modified strain of Chlorella vulgaris results in reduced green pigmentation in the modified strain of Chlorella vulgaris as compared to the wild-type strain of Chlorella vulgaris. It will be appreciated that such a modified strain of Chlorella vulgaris having reduced chlorophyll content and consequently reduced green pigmentation, as compared to the wild-type strain of Chlorella vulgaris, will be associated with a different colour than the green colour of the wild-type strain of Chlorella vulgaris. Beneficially, the modified strain of Chlorella vulgaris having the reduced chlorophyll content is a potential ingredient in various food and personal care applications. Furthermore, the reduced chlorophyll content of the modified strain of Chlorella vulgaris is also associated with reduction in the unpleasant smell or fragrance and taste or flavour associated with the wild-type strain of Chlorella vulgaris, when used in the food and personal care applications.

Optionally, the modified strain of Chlorella vulgaris has a lutein content lower than the lutein content of the wild-type strain of Chlorella vulgaris. Lutein is a primary xanthophyll (carotenoid) in green microalgae such as Chlorella vulgaris. Lutein enables the microalgae to absorb blue light and reflects yellow or orange-red light. Furthermore, the lutein functions as a light energy modulator in Chlorella vulgaris and serves as a non-photochemical quenching agent that protects cells of the Chlorella vulgaris from photochemical damage caused by high intensity of light during photosynthesis. The average normal amount of lutein in our wild-type strains is 5 mg/g dry cell weight (DCW). Moreover, the lutein content in Chlorella vulgaris is determined by genetics and regulated by conditions of growth that the Chlorella vulgaris is subjected to, including but not limited to temperature, pH of growth medium, exposure to light, nitrogen content in the growth medium or atmosphere, salinity of growth medium, rate of growth and so forth. In an example, exposure of the Chlorella vulgaris to very high temperatures and/or very high light intensity reduces the lutein content of the Chlorella vulgaris.

Optionally, the modified strain of Chlorella vulgaris has a lutein content in a range of 3 to 10 mg/g DCW when grown under heterotrophic conditions.

Optionally, the modified strain of Chlorella vulgaris has a lutein content lower than the lutein content of the wild-type strain of Chlorella vulgaris, normally in a range of 3 to 10 mg/g DCW when grown heterotrophically. For example, the lutein content of the modified strain of Chlorella vulgaris may be 3, 3.5, 4, 4.5, 5, 5.5, 6, 6.5, 7, 7.5, 8, 8.5, 9 or 9.5 mg/g DCW up to 3.5, 4, 4.5, 5, 5.5, 6, 6.5, 7, 7.5, 8, 8.5, 9, 9.5 or 10 mg/g DCW. Optionally, the modified strain of Chlorella vulgaris has a lutein content below 9 mg/g DCW, more optionally below 8 mg/g DCW, yet more optionally below 7 mg/g DCW, yet more optionally still below 6 mg/g DCW, yet more optionally still below 5 mg/g DCW, yet more optionally below 4 mg/g DCW, yet more optionally still below 3 mg/g DCW, yet more optionally still below 2 mg/g DCW, yet more optionally still below 1 mg/g DCW, and yet more optionally up to 0.1 mg/g DCW of the lutein content of a wild-type strain of Chlorella vulgaris. Notably, lower lutein content of the modified strain of Chlorella vulgaris renders the modified strain of Chlorella vulgaris to be more commercially acceptable for certain applications. For example, a modified strain of Chlorella vulgaris with a lutein content of 0.01 mg/g DCW will be more commercially acceptable in industries that require no colour in the final manufactured products, as compared to the modified strain of Chlorella vulgaris with lutein content of 1 mg/g DCW.

The reduced lutein content in the modified strain of Chlorella vulgaris results in reduced orange-red pigmentation in the modified strain of Chlorella vulgaris as compared to the wild-type strain of Chlorella vulgaris. In concert with the reduced chlorophyll content in certain strains this can result in a genetically determined colour form of Chlorella vulgaris with lime, pale green or even a white appearance when grown under the same conditions as the wild-type strain. Beneficially, the modified strain of Chlorella vulgaris having the reduced lutein content is a potential ingredient in various food and personal care applications.

The content of chlorophyll a, chlorophyll b and/or lutein and/or other pigments in the modified strain of Chlorella vulgaris can be determined using analytical methods known to the skilled person, for example chromatographic or spectrophotometric techniques.

Furthermore, the modified strain of Chlorella vulgaris maintains a minimum protein content of 20%, or optionally 25%, or optionally 30%, or optionally 35% protein, or optionally 40% w/w, or optionally 45% w/w, and still more optionally 50% w/w. For example, the protein content of the modified strain of Chlorella vulgaris may be from 20%, 25%, 30%, 35%, 40% or 45% up to 30%, 35%, 40%, 45% or 50% w/w. It will be appreciated that it is possible that some strains may be used to produce biomass with more than 50% w/w of protein content.

Briefly, Chlorella vulgaris strains were grown in 20 millilitre (ml) of liquid medium containing glucose or acetate at a starting cell density of 2×10⁶ cells/ml. Cells were grown in the dark at 26° C. for 6 days. A 10 ml aliquot was removed and centrifuged (4500×g, 10 minutes) to collect the cells; the pellets were washed in 1 ml double-distilled (dd) H₂O and centrifuged again (4500×g, 10 minutes). The resulting biomass pellets were dried by lyophilisation in pre-weighed tubes. Once dry, the dry cell weight (DCW) was determined before carrying out the extraction.

To extract the chlorophyll, 1 ml of methanol:acetone (1:1) was added to each sample, samples were then mixed by vortexing and pelleted (4500×g, 5 minutes). The supernatants were collected into separate tubes. This was repeated 4 times for each sample with each supernatant pooled with previous until 5 ml was collected for each sample. To complete the extraction 1 ml of dichloromethane:methanol (1:3) was added to the pellet and the previous step repeated and the 1 ml supernatant added to the previous 5 ml. This was followed by 1 ml dichloromethane to yield a total of 7 ml total supernatant. To extract any residual pigment remaining in biomass pellets after all above extraction steps had been performed, 1 ml dichloromethane:methanol (1:1) was added to the samples with 500 micrometre (μm) glass beads and sonicated for 10 minutes and the supernatant was again added to the previous 7 ml. The extractions were carried out in low light conditions-samples were wrapped in foil between processing steps to protect any pigments from degradation by light or chemical reactions catalysed by light.

The extracted pigments were dried at 60° C. by evaporation and dried pellets were resuspended in 80% acetone. Absorbance was measured spectrophotometrically at 647, 664, and 750 nanometers (nm).

The following formulas were used to estimate chlorophyll content as described in Porra et al. (1989; DOI: 10.1016/S0005-2728 (89) 80347-0).

$$Chlorophyll a = ( 12.25 \times ( A 664 - A 750 ) ) - ( 2.55 * ( A 647 - A 750 ) )$$

$$Chlorophyll b = ( 20.31 \times ( A 647 - A 750 ) ) - ( 4.91 * ( A 664 - A 750 ) )$$

$$Total chlorophyll = ( 17.76 \times ( A 647 - A 750 ) ) + ( 7.43 * ( A 664 - A 750 ) )$$

All strains were analysed in biological triplicate.

Optionally, the method 100 incorporates thin layer chromatography to separate and visualise the pigment composition in different strains. Chlorella vulgaris strains were grown in 20 ml of liquid growth medium containing 1% glucose. Cells were grown in the dark at 26° C. for 6 days. A 10 ml aliquot was removed and the cells were collected by centrifugation (4500×g, 10 minutes).

To extract the pigments, 0.5 ml of dichloromethane:methanol (1:1) was added to each sample, samples were then mixed by vortexing and centrifuged again (21000×g, 5 minutes). The supernatants, containing the extracted pigments, were collected in separate collection tubes. This extraction was repeated on the pellet 2 times and pooled into the same collection tube each time for each sample.

The entire extraction process was carried out in low light laboratory conditions (<50 μmol/m²/s), samples were wrapped in aluminium foil between processing steps.

Small quantities of the samples were deposited on a Silica gel on TLC Alu foil plate (91835-50EA, Sigma-Aldrich®) and developed with a solvent solution of 5:3:2 Hexane:EthylAcetate:acetone. Optionally, the solvent solution comprises 85% methanol in water with 0.5% trifluoroacetic acid. Resultant plates were imaged to record the separation and relative composition of pigments for each sample.

Optionally, the method 100 incorporates cell sorting by flow cytometry as an enrichment step to sort chlorophyll-deficient cells away from wild-type cells based upon the relative signal strength of autofluorescence. Typically, a sample containing cells is suspended in a fluid and injected into a flow cytometer instrument, wherein the flow of the sample is set at one cell at a time. The flow rate of the flow cytometer instrument may be any suitable rate. Optionally, the flow rate of the flow cytometer instrument is 60000 to 500000 events per minute. The flow rate may also be lower than 60000 or higher than 500000 events per minute. The flow cytometer employs lasers of various wavelengths for multi-parametric analysis of the cells in a heterogenous cell population. The light scattered by the cell is a characteristic of the cell and components therein. Typically, cells are labelled with fluorescent markers to enable first absorption of light and later its emission in a band of wavelengths.

Optionally, a 488 or 561 nm or 640 nm laser is used to elicit strong chlorophyll autofluorescence from a mixture of live cells (Chapman et al, 2005; DOI: 10.1016/j.pbi.2005.09.011). The photons from this chlorophyll autofluorescence can be detected with emission filters ranging from 640 to 800 nm. The population of cells that exhibit strong autofluorescence is sorted away from those cells that have null or significantly reduced signal as an enrichment step to enrich for those cells within the total population that have accumulated mutations that knock down or abolish the chlorophyll signal. This step can be applied optionally between 2-7 days post-exposure to mutagen and is applied in liquid culture. Null cells including those desired cells with reduced chlorophyll content are expanded by cultivation and re-sorted through one additional round to confirm the stability of the chlorophyll deficient phenotype. They can then be further expanded in liquid culture or plated onto agar plus glucose plates (or other organic carbon source) for scoring of colours with respect to other mutants. As a control to calibrate the cytometry, wild-type cells are extracted using 90% acetone to remove chlorophyll and are then photo-bleached using strong light for 20-30 minutes. These chlorophyll null cells are then used to calibrate the sorter with regard to chlorophyll deficient particles. Further, flow cytometry enables cell counting, cell sorting, determining cell characteristics and functions, detecting microorganisms, biomarker detection, protein engineering detection, and the like.

Using full spectral emission cell sorting, after excitation with a laser such as a, 488 nm, 561 nm or a 640 nm laser, multiple emission wavelengths of chlorophyll (and other carotenoids) can be measured simultaneously using greater than 60 emission filters, allowing cells exhibiting subtle shifts in total chlorophyll concentration and specific chlorophyll and carotenoid concentrations to be differentiated and sorted based on changes in the full emission spectrum. Such cells can be further enriched and expanded as described hereinabove. Specifically, the population of cells that exhibit strong autofluorescence is sorted away from those cells that have null or significantly reduced signal as an enrichment step to enrich for those cells within the total population that have accumulated mutations that knock down or abolish the chlorophyll signal. This step can be applied optionally between 2-7 days post-exposure to mutagen and is applied in liquid culture. Null cells including those desired cells with reduced chlorophyll content or desired carotenoid content are expanded by cultivation and re-sorted through one additional round to confirm the stability of the chlorophyll deficient phenotype. They can then be further expanded in liquid culture or plated onto agar plus glucose plates (or other organic carbon source) for scoring of colours with respect to other mutants.

Optionally, once expanded, the chlorophyll deficient cell population that is actively growing, can be sorted into sub-populations or single cells using the application of different lasers exciting at specific wavelengths and concurrent detection of deflection of the laser beam and specific fluorescence emissions of higher wavelength photons from cellular compounds, which can be used to differentiate specific pigment combinations that would ultimately influence the resultant stable biomass colour for a given biomass that is derived from a particular population of cells or single cells carrying specific genotype.

Optionally, the method 100 further comprises filtering out unhealthy colonies of the modified strain of Chlorella vulgaris. It will be appreciated that during mutagenesis of the wild-type strain of Chlorella vulgaris, cells of the modified strain of Chlorella vulgaris may acquire mutations at multiple sites within the genome, including a mutation or mutations that are causative for the desired phenotype. However, some cells of the modified strain of Chlorella vulgaris may additionally acquire deleterious mutations corresponding to one or more undesired phenotypes, for instance in essential genes. In such an instance, it is essential to filter out these unhealthy cells of the modified strain of Chlorella vulgaris associated with the deleterious mutations, to ensure selection of only those cells that are robust and able to grow well under desired cultivation conditions. This can be achieved by cultivation of the organisms during the period immediately following exposure to the chemical or physical mutagen under stressed or less permissive (or non-permissive) conditions, for instance at the limit of or slightly above the normal upper temperature for cultivation and in the absence of light but in the presence of glucose. Only robust strains are able to proliferate under these conditions. This approach enriches for strains that are not compromised in their replication capacity. Furthermore, after cultivation in the dark, the desired phenotypes related to colour can be scored. In such an example, the desired phenotype of the modified strain of Chlorella vulgaris is associated with white, cream, pale yellow, yellow, pale green, golden, caramel, orange, pink, red, brown or lime colour. Undesired colonies will be associated with other colours including the wild-type, dark green colour and are not selected. In other words, they are filtered out. Optionally, cells of modified strain of Chlorella vulgaris that exhibit the desired phenotype across a series of generations are selected as healthy colonies. More optionally, the mutated strain of Chlorella vulgaris is cultivated at a temperature that is slightly higher than an ideal temperature (such as, above 28° C.) for cultivation of the microalgal strain, to select only healthy cells of the modified strain of Chlorella vulgaris.

Optionally, colonies of the modified strain of Chlorella vulgaris associated with the desired phenotypes are identified, isolated and streaked sequentially and iteratively on a solid medium to obtain pure isogenic strains as well as to assess the stability of the colour phenotype under conditions more approximating a commercial cultivation scheme. The pure strains are further inoculated using a liquid media. Optionally, the liquid media may be at least one of TAP (Tris-Acetate-Phosphate), High Salt Medium™ (HSM™) plus glucose (for example, having 1% w/v glucose). More optionally, the pure strains are cultivated in dark conditions at the specific temperature of 25° C. (or between 2° and 35° C.) for 1-3 weeks and monitored over multiple successive generations for stable phenotypes. Such stable phenotypes may be associated with a lack of green colour within the modified strain of Chlorella vulgaris and/or the presence of white, cream, pale yellow, yellow, pale green, golden, caramel, orange, pink, red, brown or lime colour phenotypes.

The modified strains of Chlorella vulgaris may be subjected to additional rounds of mutagenesis. Optionally, the method 100 further comprises performing steps (b) to (d) repeatedly for selecting healthy variants of the modified strain of Chlorella vulgaris based on desired traits, wherein the desired traits comprise a colour, a pigment content, a protein content and improved tolerance to process conditions selected from a group of temperature, pH, sheer stress and osmolality. Furthermore, the Chlorella vulgaris strains are genetically stable.

Sequential, successive mutagenesis of Chlorella vulgaris may be followed by cultivating the mutated strains of Chlorella vulgaris at a specific temperature, for a predefined period of time, without presence of light and in the presence of an organic carbon energy source, and identifying cells of the mutated strain of Chlorella vulgaris having a colony phenotype (or desired trait) different from the parental strain of Chlorella vulgaris. Optionally, the method 100 incorporates cell sorting by flow cytometry to sort cells based on the desired traits. However, the selection for desired traits may be achieved using any of the disclosed methods or methods available to those skilled in the art. Desired traits to include, but not limited to, pigment content, colour, protein content, other macromolecular content and improved tolerance to process conditions including but not limited to cultivation temperature, pH, sheer stress and osmolality.

Furthermore, the modified strain of Chlorella vulgaris of the invention is genetically stable. The term “genetically stable” as used herein, refers to a characteristic of a species or a strain/isolate to resist changes and maintain its genotype over multiple generations or cell divisions, ideally hundreds to thousands. In an example, the genetically stable strains of Chlorella vulgaris are genetically determined to produce one of: white, cream, pale yellow, yellow, pale green, golden, caramel, orange, pink, red, brown or lime colour, determined by lack of or reduced production of chlorophyll and/or other pigments in the modified strain of Chlorella vulgaris, and in some instance production of increased amounts of intermediate pigments or new pigmented chemical products resulting from the underlying genetic changes. The wild-type strains of Chlorella vulgaris are haploid. This is important because it was observed that when diploid strains of other Chlorella species or closely related species were used to derive colour phenotype mutants, a rapid change in the phenotype, for example reverting back from a desired yellow colour to green, was observed over several generations of cultivation. This indicates the relative instability of the desired phenotype in diploid microalgal strains other than confirmed haploid Chlorella vulgaris strains. Optionally, the modified strain of Chlorella vulgaris is genetically stable with respect to the observed colour phenotype. Notably, the visual inspection of strains of Chlorella vulgaris maintained both on agar and in liquid culture is sufficient to conclude that the phenotype, such as colour, is genetically stable in the modified strain of Chlorella vulgaris.

Another embodiment of the present disclosure provides an algae biomass derived from a modified strain of Chlorella vulgaris having a chlorophyll content lower than the chlorophyll content of a wild-type strain of Chlorella vulgaris grown under the same conditions, or obtained by performing a method 100 of producing a modified strain of Chlorella vulgaris having a chlorophyll content lower than the chlorophyll content of a wild-type strain of Chlorella vulgaris grown under the same conditions.

Furthermore, disclosed is a composition comprising an algae biomass derived from a modified strain of Chlorella vulgaris having a chlorophyll content in a range of 0.001 to 0.5 mg/g of DCW. Also disclosed is a composition comprising an algae biomass derived from the modified strain of Chlorella vulgaris having a chlorophyll content lower than the chlorophyll content of a wild-type strain of Chlorella vulgaris grown under the same conditions, or obtained by performing the method 100 of producing the modified strain of Chlorella vulgaris. In this context, the term “algae biomass” as used herein, refers to biomass derived from algae (microalgae or macroalgae) that is cultivated heterotrophically. Alternatively, modified strain of Chlorella vulgaris having a chlorophyll content of 5% or less, could still grow mixotrophically. The algae biomass is obtained from the modified strain of Chlorella vulgaris having a chlorophyll content in a range of 0.001 to 0.5 mg/g of DCW. Furthermore, such an algae biomass can be obtained by performing the method 100 of producing the modified strain of Chlorella vulgaris (as explained in detail hereinabove). Optionally, the algae biomass can be obtained from the modified strain of Chlorella vulgaris under current good manufacturing practice (CGMP) conditions. Optionally, the algae biomass derived from the modified strain of Chlorella vulgaris has a chlorophyll content of 0.50 to 0.25 mg/g DCW, preferably 0.25 to 0.10 mg/g DCW, and most preferably 0.1 to 0.001 mg/g DCW.

Optionally, the composition may be a food or food ingredient. The term “food” refers to an edible product that can be directly or indirectly (such as, subsequent to preparation) consumed by humans and/or animals. The term “food ingredient” refers to a substance incorporated into food during one of: production, processing, treatment, packaging, transportation, distribution, preservation, storage and so forth of food. Optionally, the food ingredients are incorporated into the food to improve and/or maintain freshness, nutritional value, appearance, texture, taste and/or safety of the food.

Optionally, the composition is a cosmetic or cosmetic ingredient. The term “cosmetic” refers to a substance or product used to enhance or alter the appearance or texture of a body part (such as face or skin) or fragrance by direct or indirect application on body (humans and/or animals). The cosmetic(s) is generally a mixture of chemical compounds derived from natural sources (such as herbs), synthetic sources (such as chemicals) or a mixture thereof. Cosmetic(s) include, but do not limit to, lipsticks, mascara, kohl, eye liner, eye shadow, foundation, blush, highlighter, bronzer, creams and perfumes. The term “cosmetic ingredient” refers to a substance incorporated into cosmetics during one of: production, processing, treatment, packaging, transportation, distribution, preservation, storage and so forth of cosmetics. Optionally, the cosmetic ingredients are incorporated into the cosmetic to improve and/or maintain freshness, product value, appearance, texture, flavour, fragrance and/or safety thereof.

Optionally, the composition comprising algae biomass derived from the aforementioned modified strain of Chlorella vulgaris or obtained by performing the aforementioned method 100 is employed as an ingredient in at least one of: human foods, human nutraceutical preparations or formulations, animal feeds, drug compositions, cosmetics, personal care compositions, personal care devices, or textiles, dyes or inks. It will be appreciated that the reduced chlorophyll content and optionally, the reduced lutein content provides the modified strain of Chlorella vulgaris one or more parameters such as appealing appearance, pleasant smell (fragrance) and/or taste (flavour). Consequently, such one or more parameters of the modified strain of Chlorella vulgaris enable usage in various products used or consumed by humans and/or animals. For example, the algae biomass obtained from the modified strain of Chlorella vulgaris can be used as an ingredient in food for humans, animal feed, nutraceutical preparations and formulation for humans, pharmaceutical compositions, cosmetics, personal care compositions, personal care devices, textiles, dyes or inks, and so forth. The food for humans can include but is not limited to bakery products, pastas, cereals, cereal bars, confections, sauces, soups, frozen desserts, ice creams, cheeses, plant-based meats, yoghurts, smoothies, creams, spreads, salad dressings, mayonnaises, food garnishing and seasoning, candies, gums, jellies, vape liquid and so forth. The nutraceutical preparations and formulation comprise, for example, nutritional supplements, hormone tablets, digestive capsules, tablets, powders, oils and the like. The cosmetic formulations may employ use of the algae biomass or specific extracts derived therefrom, for example, in lipsticks, powders, creams, exfoliants, facial packs, and so forth. The personal care compositions and personal care devices comprise toothpastes, mouthwash, hand-wash, body-wash, body soaps, shampoos, oils, sun-creams, after-sun creams, sunblock and so forth. The pharmaceutical compositions include any type of compositions known to the skilled person for the delivery of medicaments, including bioactives, vaccines and other recombinant proteins and enzymes.

Optionally, there is provided a method of using the algae biomass of the invention as an ingredient in at least one of: human foods, human nutraceutical preparations or formulations, animal feeds, pharmaceutical compositions, cosmetics, personal care compositions, personal care devices, textiles, and dyes or inks. The method of use comprises using the algae biomass ingredient comprising the modified strain of Chlorella vulgaris as any one of: dried powder, dried flakes, frozen paste, an extract (an aqueous or a polysaccharide extract), solutions, suspensions, solution preconcentrates, emulsions, emulsion pre-concentrates, concoction, tablets, pills, pellets, capsules, caplet, concentrates, granules, and so forth. Furthermore, a dried, fresh, or frozen part of the modified strain of Chlorella vulgaris, oil derived from the modified strain of Chlorella vulgaris, a homogenate, whole cell, lysed cell and so forth can be used in preparation of human foods, human nutraceutical preparations or formulations, animal feeds, pharmaceutical compositions, cosmetics, personal care compositions, personal care devices, textiles, dyes or inks. Moreover, there is provided a method of using the algae of the invention as an ingredient in various food, personal care, medicinal and nutritional applications comprise combining the food or food ingredient with one or more additional edible or suitable ingredients, such as milk, oil, cream, water, spices, herbs, minerals, proteins, one or more chemical compounds, preservatives, aromatic juices, and the like, to obtain the desired human foods, human nutraceutical preparations or formulations, animal feeds, pharmaceutical compositions, cosmetics, personal care compositions, personal care devices, textiles, dyes or inks.

The modified strain of Chlorella vulgaris can be used to prepare compositions in any way known to the skilled person.

Furthermore, disclosed is a microalgae product or flour comprising a homogenate of microalgae biomass derived from the modified strain of Chlorella vulgaris of the invention, or obtained by performing the method 100 of producing the modified strain of Chlorella vulgaris. The term “microalgae flour” is used to refer to an edible composition comprising a plurality of particles of algae biomass. Optionally, the plurality of particles of algae biomass is any one of: whole cells, lysed cells or a mixture thereof. More optionally, the microalgae flour comprises one or more of significant digestible proteins, dietary fibre content, associated water binding attributes, healthy oil delivering attributes, spices, herbs, a flow agent, an antioxidant and so forth. It may be appreciated that the microalgae flour lacks visible oil and is preferably in a powdered form. The microalgae flour comprises the homogenate of microalgae biomass derived from the modified strain of Chlorella vulgaris. Furthermore, the microalgae flour is obtained by performing the method 100 of producing the modified strain of Chlorella vulgaris (as described in detail hereinabove). The microalgae flour can be produced under current Good Manufacturing Practice (CGMP) conditions.

Furthermore, the microalgae flour finds application as the food or food ingredient in various bakery products, such as breads, rolls, wraps, tortillas, pastas and the like, confections, such as cakes, pastries, chocolates, jellies, gums and so on, dairy products or non-dairy substitute products, such as yoghurt, creams, sour creams, and other food products, such as soups, sauces, seasonings and the like. Beneficially, the microalgae flour of the invention does not impart the green colour, unpleasant smell or taste associated with the wild-type strain of Chlorella vulgaris, to the processed food product. Beneficially, the modified strain of Chlorella vulgaris of the present disclosure is a natural and not genetically modified (non-GM) food source. Additionally, the modified strain of Chlorella vulgaris is nutritious (comprising high protein and fibre content), gluten free and animal-free (vegetarian and/or vegan).

Furthermore, the modified strain of Chlorella vulgaris is genetically stable and can be grown under heterotrophic growth conditions, and over time with the desired phenotype including improved colour, smell and taste parameters that are suitable for human and/or animal consumption. Consequently, such modified strains of Chlorella vulgaris may find potential applications as whole food or as an ingredient in human (and animal) foods, nutraceutical preparations, cosmetic formulations, medicines, personal care, and so on, owing to increased market uptake/acceptance.

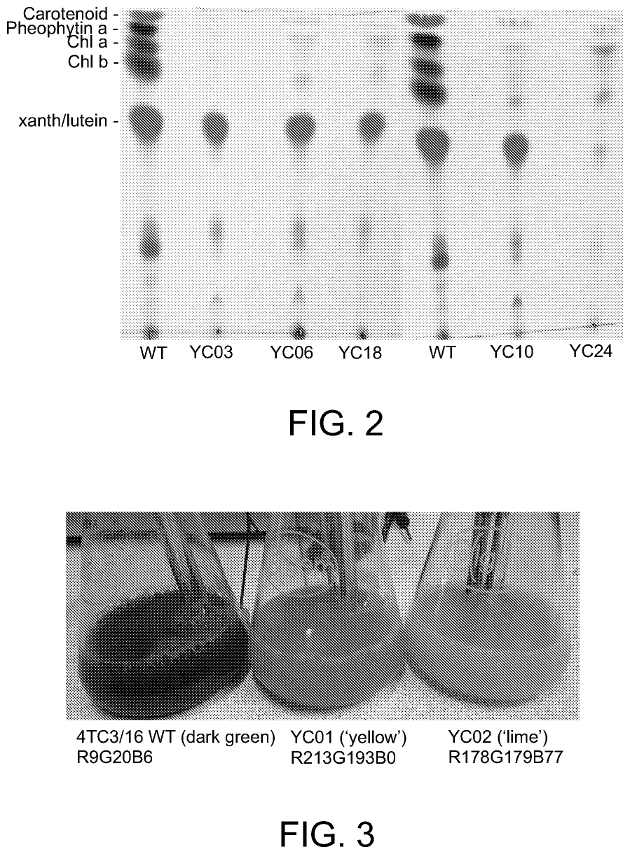

Referring to FIG. 2, there is shown a thin-layer chromatography analysis of pigments in a panel of wild-type (WT) and colour mutants. WT displays 4TC3/16 parental strains, wherein WT is a wild-type strain (parental strain) of Chlorella vulgaris (4TC3/16) and YC03, YC06, YC18, YC 10 and YC24 shows various modified strains of Chlorella vulgaris. YC03 shows a yellow strain, YC06 displays red colour, YC18 shows green/orange colour, YC10 displays brown/orange colour and YC24 displays lime colour. Notably, WT strains exhibit presence of carotenoid, pheophytin a, chlorophyll a (Chl a), chlorophyll b (Chl b) and xanthophyll and/or lutein content. However, modified strains of Chlorella vulgaris exhibit an absence of or significant reduction in chlorophyll a (Chl a) and chlorophyll b (Chl B) content and may also exhibit stable absence of or significant reduction in levels of carotenoids, xanthophylls and/or pheophytin.

Referring to FIG. 3, there is shown a comparison of wild-type (WT), yellow (YC01) and lime (YC02) Chlorella vulgaris strains cultivated in liquid culture. As shown, a spectrum of colour variants ranging from red (far left; darkest) to white (far right, lightest) is identified for modified strains of Chlorella vulgaris. It will be appreciated that colours are noted under each flask as approximate RGB values and are shown as examples only and are not intended to limit the repertoire of colours to these exact values only, i.e. R9G20B6 for WT, R13G193B0 for YC01 and R178G179B77 for YC02. Furthermore, colours demonstrated for each strain are genetically stable with the colours presented herein being the product of 2 weeks cultivation after reaching stationary phase. Colours are stable and representative of both cultivation conditions as well as the genetics of each variant strain.

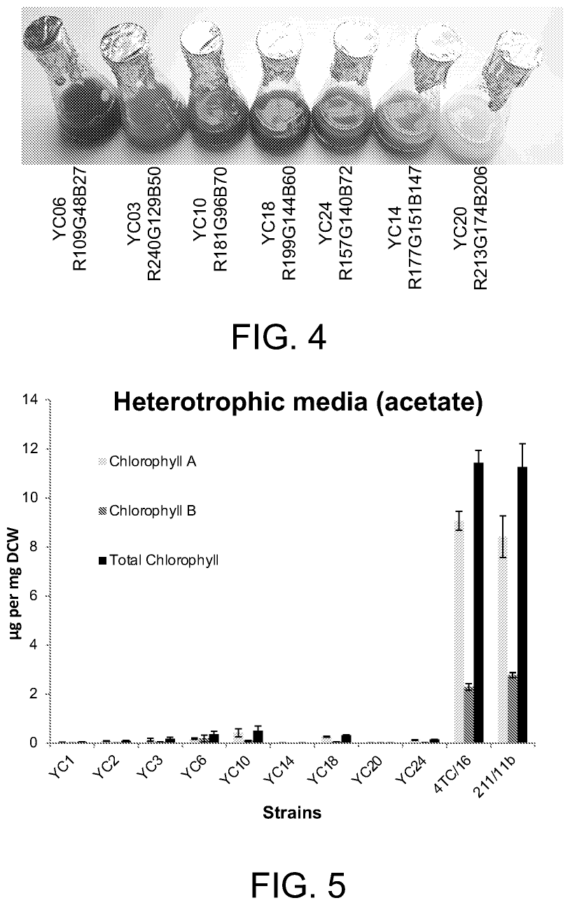

Referring to FIG. 4, there is shown a comparison of various modified strains Chlorella vulgaris, i.e. YC06, YC03, YC10, YC18, YC24, YC14 and YC20, cultivated in liquid culture. As shown, a spectrum of colour variants is identified for different modified strains of Chlorella vulgaris, YC06, YC03, YC10, YC18, YC24, YC14 and YC20. It will be appreciated that colours are noted under each flask as approximate RGB values, i.e. R109G48B27 for YC06, R240G129B50 for YC03, R181G96B70 for YC10, R199G144B60 for YC18, R157G140B72 for YC24, R177G151B147 for YC14 and R213G174B206 for YC20. Furthermore, colours demonstrated for each strain are genetically stable with the colours presented herein being the product of 2 weeks cultivation after reaching stationary phase.

Referring to FIG. 5, there is shown chlorophyll content in chlorophyll deficient colour variants as compared to the chlorophyll content produced in wild-type cells for the parental strain (4TC3/16) and a comparative, well characterised culture collection strain of Chlorella vulgaris (CCAP 211/11b) cultivated under the same conditions. The relative amounts of chlorophyll a, chlorophyll b and total chlorophyll calculated are represented in mg/g DCW when grown under heterotrophic conditions using acetate as the primary carbon source.

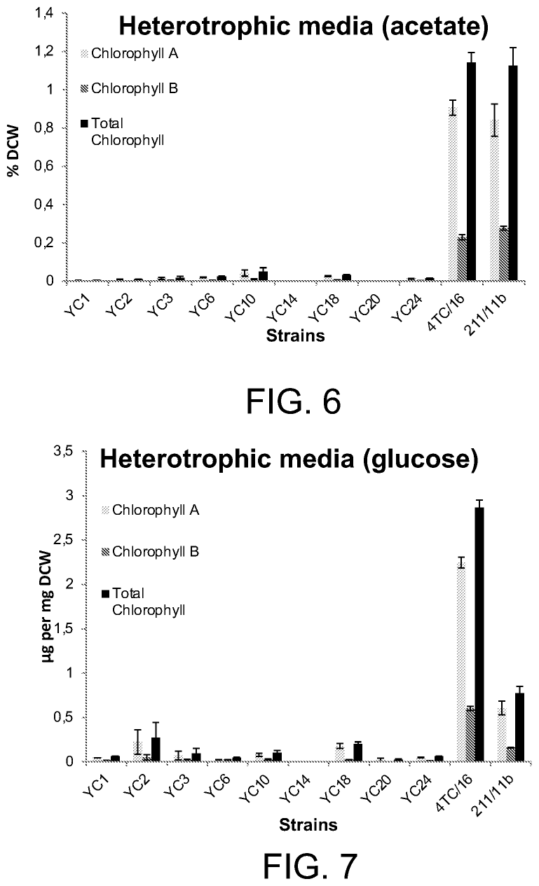

Referring to FIG. 6, there is shown chlorophyll content in chlorophyll deficient colour variants as compared to the chlorophyll content produced in wild-type cells for the parental strain (4TC3/16) and a comparative, well characterised culture collection strain of Chlorella vulgaris (CCAP 211/11b) cultivated under the same conditions. The relative amounts of chlorophyll a, chlorophyll b and total chlorophyll calculated are represented in % DCW when grown under heterotrophic conditions using acetate as the primary carbon source.

Referring to FIG. 7, there is shown chlorophyll content in chlorophyll deficient colour variants as compared to the chlorophyll content produced in wild-type cells for the parental strain (4TC3/16) and a comparative, well characterised culture collection strain of Chlorella vulgaris (CCAP 211/11b) cultivated under the same conditions. The relative amounts of chlorophyll a, chlorophyll b and total chlorophyll calculated are represented in mg/g DCW when grown under heterotrophic conditions using glucose as the primary carbon source.

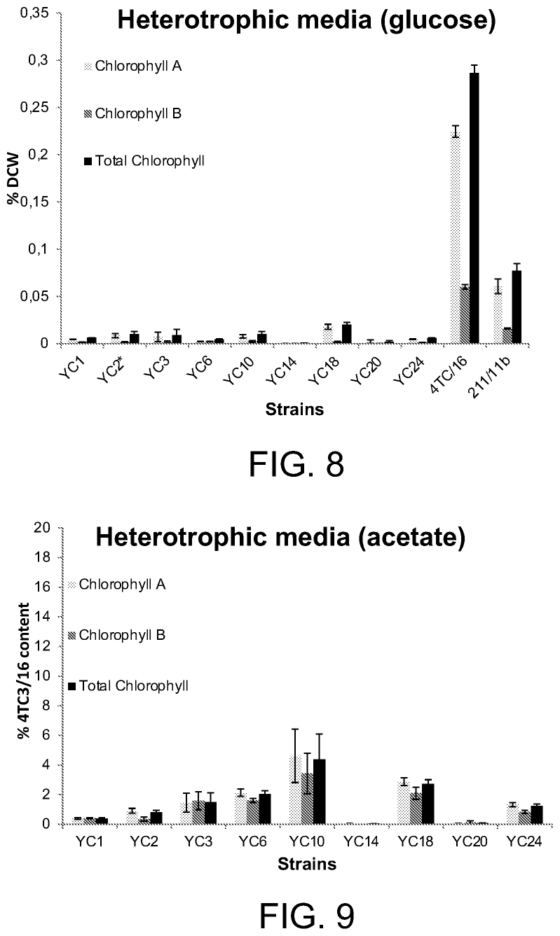

Referring to FIG. 8, there is shown chlorophyll content in chlorophyll deficient colour variants as compared to the chlorophyll content produced in wild-type cells for the parental strain (4TC3/16) and a comparative, well characterised culture collection strain of Chlorella vulgaris (CCAP 211/11b) cultivated under the same conditions. The relative amounts of chlorophyll a, chlorophyll b and total chlorophyll calculated are represented in % DCW when grown under heterotrophic conditions using glucose as the primary carbon source.

Referring to FIG. 9, there is shown chlorophyll content in chlorophyll deficient colour variants represented as a percentage of chlorophyll produced in wild-type cells cultivated under the same conditions. The relative amount of chlorophyll a, chlorophyll b and total calculated is represented in relation to the amount produced by the parental (wild-type) strain, 4TC3/16 grown under heterotrophic conditions using acetate as the primary carbon source.

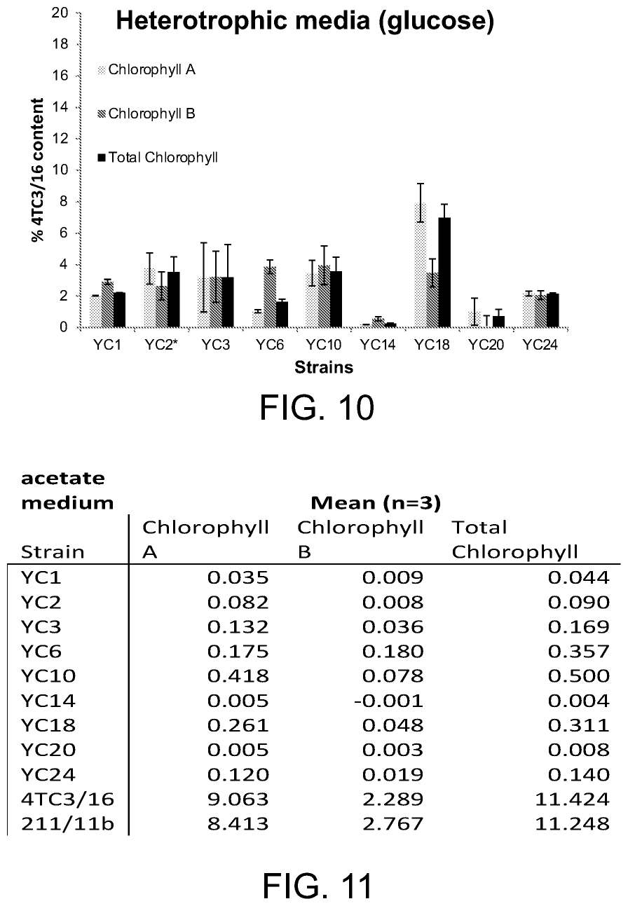

Referring to FIG. 10, there is shown chlorophyll content in chlorophyll deficient colour variants represented as a percentage of chlorophyll produced in wild-type cells cultivated under the same conditions. The relative amount of chlorophyll a, chlorophyll b and total calculated is represented in relation to the amount produced by the parental (wild-type) strain, 4TC3/16 grown under heterotrophic conditions using glucose as the primary carbon source.

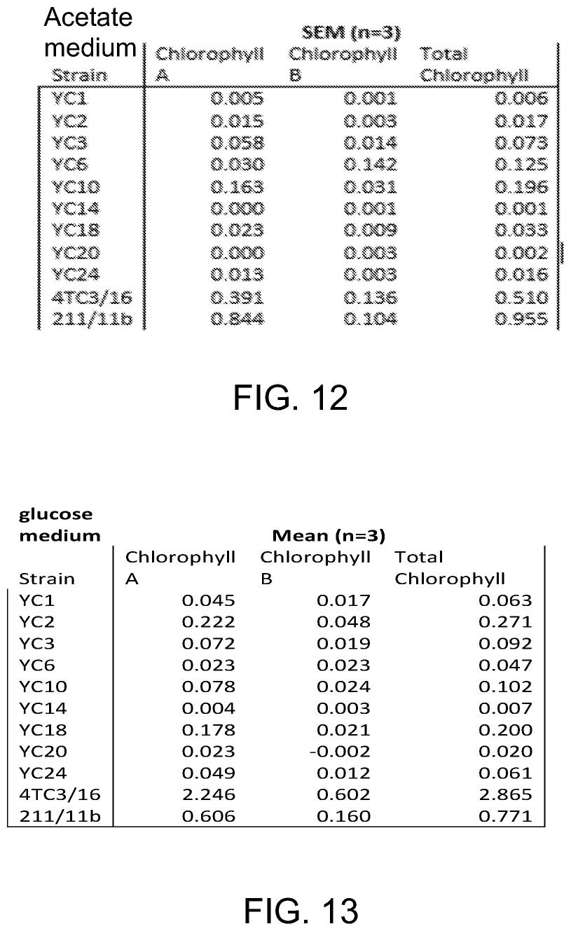

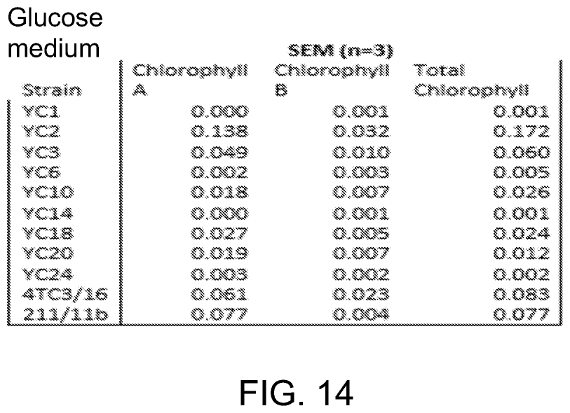

Referring to FIGS. 11, 12, 13 and 14, there is shown table of chlorophyll content of Chlorella vulgaris colour variants and wild-type cells, grown under the same conditions, is represented as the average of three measurements and expressed in microgram units. SEM, standard error of the mean.

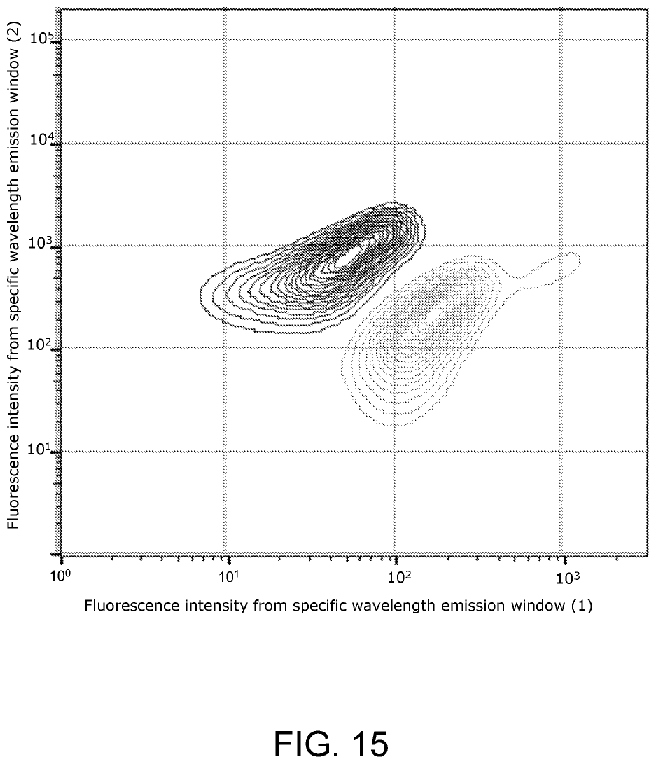

Referring to FIG. 15, there is shown an example of the use of flow cytometry to identify subpopulations of cells (white YC20 strain and yellow YC03 strain) based on pigment composition. As shown, the Y-axis depicts the fluorescence intensity from a specific wavelength emission window (2) following excitation at a specific wavelength (2) and the X-axis depicts the fluorescence intensity from a specific wavelength emission window (1) following excitation at a specific wavelength (1). The representative population from the white YC20 strain is shown in black contours, while a representative cell population from the yellow YC03 strain is shown in grey contours.

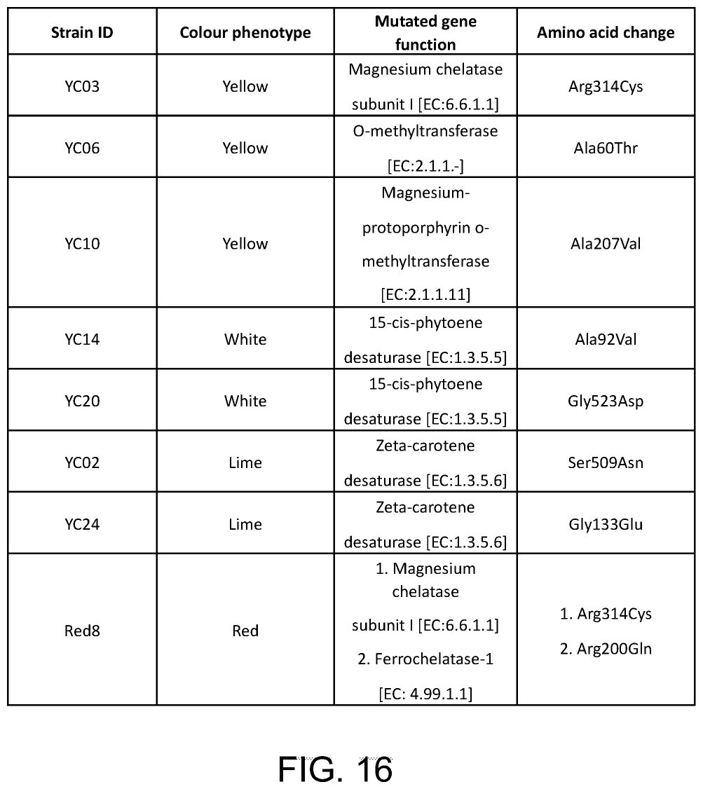

Referring to FIG. 16, there is shown a table of chlorophyll-deficient EMS mutants of Chlorella vulgaris 4TC3/16 (wild-type) strain, in accordance with an embodiment of the present disclosure. Chlorella vulgaris 4TC3/16 (wild-type) was exposed to a titrated quantity of ethyl methanesulphonate (EMS) mutagen for a predefined duration and chlorophyll-deficient mutants of Chlorella vulgaris 4TC3/16 were visually identified by their colour on agar plates, following cultivation of mutant pools generated by EMS mutagenesis, under heterotrophic conditions. In accordance with another embodiment of the present disclosure, a modified strain of Chlorella vulgaris was exposed to one or more additional rounds of mutagenesis using a titrated quantity of ethyl methanesulphonate (EMS) mutagen for a predefined duration and additional colour mutants were visually identified by their colour on agar plates, following cultivation of mutant pools generated by EMS mutagenesis, under heterotrophic conditions. The functional mutations in colour phenotypes of the chlorophyll-deficient EMS mutants of Chlorella vulgaris 4TC3/16 (wild-type) strain, namely YC02, YC24, YC03, YC06, YC10, YC14, YC20 and the Red8 strain, which is the product of two rounds of mutagenesis, were identified by comparing their genomic sequences with the wild type genomic sequence. The wild type genome was first assembled from data produced by using a combination of PacBio and short read (Illumina) data. Single nucleotide variants in the mutants were subsequently identified using short-read data (Illumina). The phenotype of each mutant can be explained by a single missense mutation attributable to EMS (or two mutations in the case of Red8) (i.e. transition of a purine to another purine nucleotide, or pyrimidine to another pyrimidine nucleotide, as referenced in Sega 1984; DOI: 10.1016/0165-1110 (84) 90007-1) in genes with a corresponding function.

The three independently isolated yellow-colour mutants of Chlorella vulgaris 4TC3/16, namely YC03, YC06 and YC10, have mutations in genes of the chlorophyll biosynthesis pathway, i.e. magnesium chelatase subunit I, O-methyltransferase, and magnesium-protoporphyrin O-methyltransferase, respectively.

As shown, the modified strain of Chlorella vulgaris YC03, exhibiting yellow colour phenotype is a result of mutation that replaces arginine with cysteine at codon 314 of enzyme magnesium chelatase subunit I [EC: 6.6.1.1] (SEQ ID NO: 1). The enzyme magnesium chelatase subunit I catalyzes insertion of magnesium ion into protoporphyrin IX to yield Mg-protoporphyrin IX. The modified strain of Chlorella vulgaris YC06, exhibiting yellow colour phenotype, is a result of mutation that replaces alanine with threonine at codon 121 of enzyme O-methyltransferase [EC: 2.1.1.-] (SEQ ID NO: 2). The modified strain of Chlorella vulgaris YC10, exhibiting yellow colour phenotype, is a result of mutation that replaces alanine with valine at codon 301 of enzyme magnesium-protoporphyrin O-methyltransferase [EC: 2.1.1.11] (SEQ ID NO: 3). The enzyme magnesium-protoporphyrin O-methyltransferase catalyzes conversion of magnesium-protoporphyrin IX to magnesium-protoporphyrin IX methylester. As a result, these yellow colour mutants cannot synthesize chlorophyll, but remain able to produce carotenoids, such as lutein, violaxanthin and neoxanthin (metabolic products of the two branches of the carotenoid biosynthesis pathway in Chlorella vulgaris.

The two independently isolated white-colour mutants of Chlorella vulgaris 4TC3/16, namely YC14 and YC20, have mutations in gene encoding for phytoene desaturase (PDS), i.e. 15-cis-phytoene desaturase, of the carotenoid biosynthesis pathway. Both mutants were shown to accumulate phytoene—the colourless substrate of PDS, using thin layer chromatography. In addition, these mutants are chlorophyll-deficient, assayed by HPLC. Although phytoene is a carotenoid precursor and not a chlorophyll precursor, both pigments are ultimately produced from geranylgeranyl diphosphate (GGPP), referred to as a “metabolic hub” for biosynthesis of numerous isoprenoids (as described in Camagna et al. 2019; DOI 10.1104/pp. 18.01026). Disruption of PDS in Arabidopsis mutants results in inhibition of genes for both chlorophyll and carotenoid biosynthesis; likely a negative feedback on gene expression causes accumulation of phytoene (Qin et al. 2007; DOI: 10.1038/cr.2007.40). Therefore, the disclosed single mutations of PDS in modified strains of Chlorella vulgaris YC14 and YC20, could be sufficient to account for both the chlorophyll and carotenoid-deficient phenotype observed in these strains.

As shown, the modified strain of Chlorella vulgaris YC14, exhibiting white colour phenotype, is a result of mutation that replaces alanine with valine at codon 92 of enzyme 15-cis-phytoene desaturase [EC: 1.3.5.5] (SEQ ID NO: 4). The enzyme 15-cis-phytoene desaturase (PDS) catalyzes conversion of phytoene into zeta-carotene via the intermediate, phytofluene. The modified strain of Chlorella vulgaris YC20, exhibiting white colour phenotype, is a result of mutation that replaces glycine with aspartic acid at codon 523 of the enzyme PDS (SEQ ID NO: 5).

The two independently isolated lime-colour mutants of Chlorella vulgaris 4TC3/16, namely YC02 and YC24, have mutations in a gene encoding for zeta-carotene desaturase (ZDS) within the carotenoid biosynthesis pathway. These mutants have a chlorophyll-deficient phenotype, and also accumulate zeta-carotene, the lime coloured substrate of ZDS, assayed by HPLC. GGPP is a precursor of the carotenoid pathway, and ZDS represents a node in this pathway.

As shown, the modified strain of Chlorella vulgaris YC02, exhibiting lime colour phenotype, is a result of mutation that replaces serine with asparagine at codon 509 of enzyme ZDS [EC: 1.3.5.6] (SEQ ID NO: 6). The enzyme ZDS catalyses conversion of zeta-carotene to lycopene via the intermediate, neurosporene. The modified strain of Chlorella vulgaris YC24, exhibiting lime colour phenotype, is a result of a mutation that replaces glycine with glutamic acid at codon 133 of the enzyme ZDS (SEQ ID NO: 7).

The isolation of independent lines exhibiting mutations in PDS and ZDS is evidence that the genetic basis of the named white colour phenotypes (YC14 and YC20) and lime colour phenotypes (YC02 and YC24) arose from mutations in a single gene. Similarly, bioinformatic analysis of independent mutations in genes affecting nodes of the chlorophyll biosynthesis pathway is sufficient to explain the observed yellow colour phenotypes (YC3, YC6 and YC10), i.e. the phenotypes of each strain have a monogenic basis; affecting a gene of an attributable function. In addition, it should be obvious from these results, that an alternate method to obtain a white colour phenotype would be to conduct one or more further rounds of mutagenesis and selection, using lime or yellow mutant strains as the parental strain, and isolating mutants with additional gene knockouts in carotenoid biosynthesis, for example.

As shown, the modified strain of Chlorella vulgaris Red8, exhibiting red colour phenotype, is a result of mutations which replace Arginine with Glutamine at position 200 in ferrochelatase-1 [EC: 4.99.1.1] (as set forth in SEQ ID NO: 8) and arginine with cysteine at codon 314 of enzyme magnesium chelatase subunit I [EC: 6.6.1.1] (as set forth in SEQ ID NO: 1). The enzyme ferrochelatase-1 catalyses the last step of heme biosynthesis by inserting ferrous iron into protoporphyrin IX to produce protoheme. Accordingly, the combination of mutations in both ferrochelatase-1 and magnesium chelatase subunit I result in accumulation of protoporphyrin IX, the precursor of both chlorophyll and heme proteins, which results in a red phototype. The Red8 strain was isolated following a second round of mutagenesis in which YC03 was used as the parent strain.

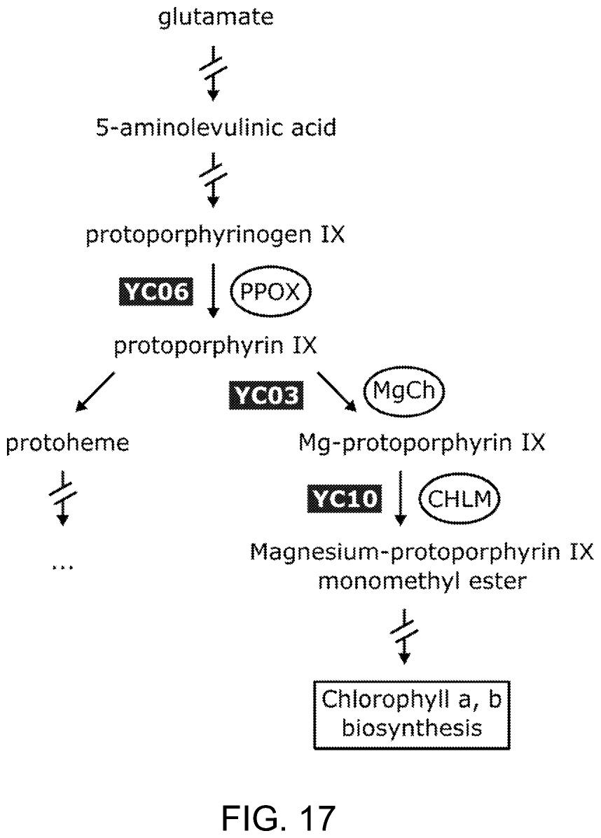

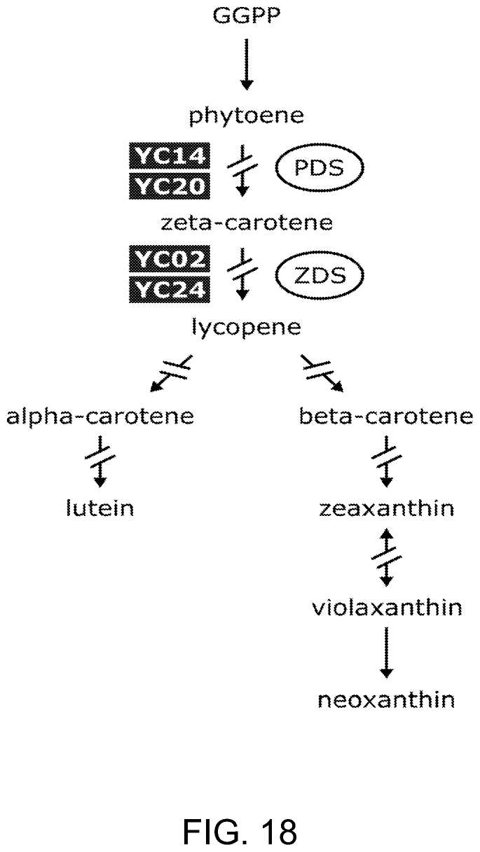

Referring to FIG. 17, there is shown an overview of tetrapyrrole and chlorophyll biosynthesis in Chlorella vulgaris, in accordance with an embodiment of the present disclosure. The position of mutated enzymes in chlorophyll-deficient mutants of Chlorella vulgaris 4TC3/16 with the yellow-colour phenotype YC03, YC06 and YC10 is shown. YC03 has a mutation affecting magnesium chelatase subunit I (MgCh), YC06 has a mutation affecting protoporphyrinogen IX oxidase (PPOX), and YC10 has a mutation affecting Mg-protoporphyrin methyl transferase (CHLM).

Referring to FIG. 18, there is shown an overview of carotenoid biosynthesis in Chlorella vulgaris, in accordance with an embodiment of the present disclosure. The position of mutated enzymes in chlorophyll- and carotenoid-deficient mutants of Chlorella vulgaris 4TC3/16 with the white-colour phenotype (YC02 and YC24) and the lime-colour phenotype (YC14 and YC20) is shown. YC20 and YC14 have mutations affecting phytoene desaturase (PDS) while YC02 and YC24 have mutations affecting zeta-carotene desaturase (ZDS). Both PDS and ZDS catalyze two consecutive desaturations of their respective substrates to produce their respective products.

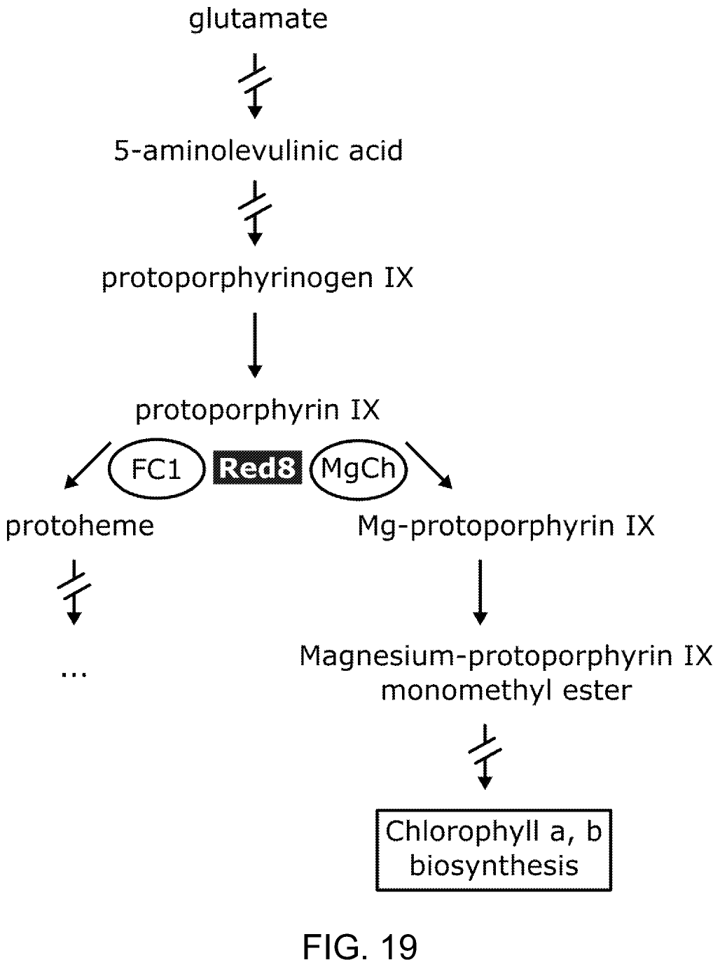

Referring to FIG. 19, there is shown an overview of chlorophyll biosynthesis in Chlorella vulgaris red variants (e.g. Red8), in accordance with an embodiment of the present disclosure. The position of mutated enzymes in a chlorophyll deficient mutant of Chlorella vulgaris exhibiting a red phenotype (Red8) are shown. Red8 has mutations affecting both magnesium chelatase subunit I (MgCh), and ferrochelatase-1 (FC1).

It will be appreciated in FIGS. 17, 18 and 19 that there may be multiple pathways involved directly or indirectly in tetrapyrrole, chlorophyll and carotenoid biosynthesis in Chlorella vulgaris, respectively. However, not all such pathways and their intermediates and metabolites may be relevant to the present disclosure. Therefore, intermediates and other metabolites not particularly relevant to the illustrated tetrapyrrole, chlorophyll and carotenoid biosynthesis pathways have been omitted for clarity, indicated by broken arrow symbols.

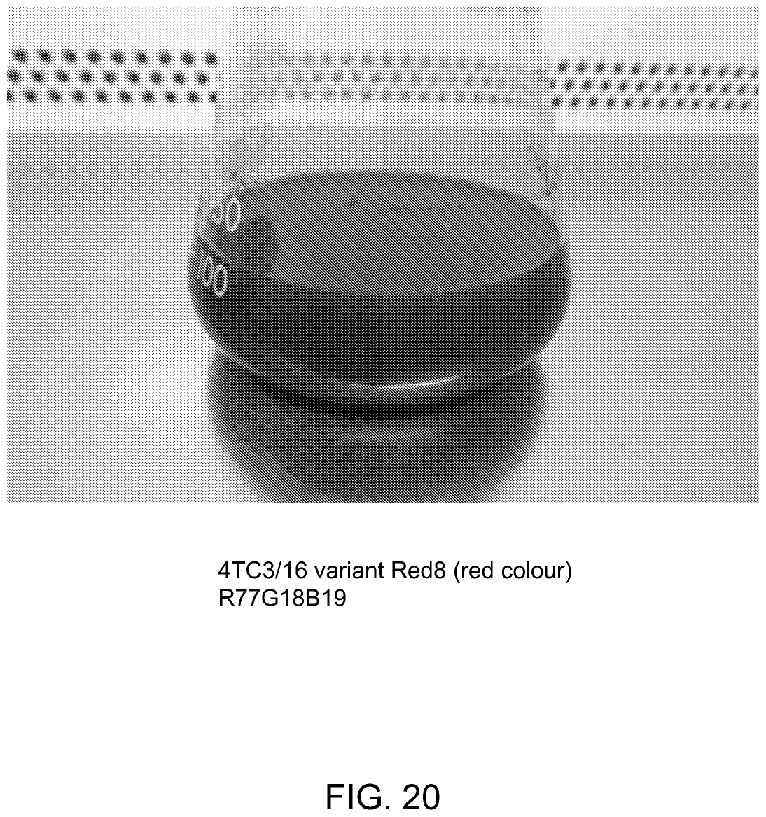

FIG. 20 shows Chlorella vulgaris red variant (Red8) cultivated in liquid culture, in accordance with an embodiment of the present disclosure. It will be appreciated that colour is noted under each flask as an approximate RGB value and is shown as an example only and is not intended to limit the repertoire of colour to this exact value only, i.e. R77G18B19 for Red8. Furthermore, the red colour demonstrated for this strain is genetically stable with the colour presented herein being the product of 2 weeks cultivation after reaching stationary phase. The colour is stable and representative of both cultivation conditions as well as the genetics of the variant strain.

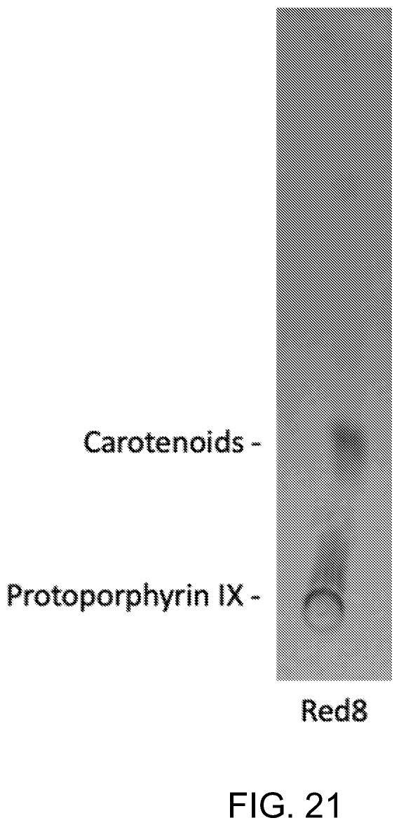

Referring to FIG. 21, there is shown a thin-layer chromatography analysis of pigments in Chlorella vulgaris red variant (Red8). Protoporphyrin IX is a red-colour porphyrin which is a precursor within the tetrapyrrole biosynthesis metabolic pathway. In the red Chlorella vulgaris variants of the invention, Protoporphyrin IX accumulates-conferring a red colour phenotype. Protoporphyrin IX is identified by TLC chromatography in a solvent extract from Red8, where it is separated from carotenoids using, for example 85% methanol in water with 0.5% trifluoroacetic acid. Notably, the Red8 pigment extraction also exhibits absence of or significant reduction in chlorophyll a (Chl a) and chlorophyll b (Chl B) content.

The present invention is advantageous as it provides a genetically stable, modified strain of Chlorella vulgaris having a chlorophyll content lower than a chlorophyll content of a wild-type strain of Chlorella vulgaris grown under the same conditions. Furthermore, the method ensures reduction in the green colour of the existing parental strains (or the wild-type strain of Chlorella vulgaris) while identifying those modified strains of Chlorella vulgaris where the genetic changes do not result in a negative impact on growth under commercial scale cultivation conditions. Moreover, any cells of the modified strain of Chlorella vulgaris that might have displayed the desired change in colour but may have also acquired deleterious mutations are filtered out, thus, providing robust, commercially scalable strains of the modified strain of Chlorella vulgaris having the desired phenotype. Furthermore, the mutagenesis conditions comprise the use of a non-lethal (minimal dosage) quantity of the mutagenic chemical, thus, enabling the derivation of the desired phenotype in the modified strain of Chlorella vulgaris while balancing against excessive accumulation of undesirable mutations that reduce cell fitness thereof. Additionally, beneficially, the method affords derivation of strains where vitality and scalability of the strains under desired fermentation conditions is improved.

Additionally, beneficially, the green microalga, Chlorella vulgaris, has been identified as a superfood and is not subject to Novel Food Regulation (EC) No. 258/97 due to the fact that it was on the market in Europe as a food or food ingredient and consumed to a significant degree before 15 May 1997. Accordingly, the organism is considered safe to eat for both humans and animals both as a whole food and as an ingredient. Furthermore, Chlorella vulgaris is included within the CIRS China list of cosmetic ingredients both as whole cell and as extract as well as being included on the European Cosmetics Ingredients list. This is in contrast to other related microalgae, specifically Chlorella sorokiniana and now Auxenochlorella (previously Chlorella) protothecoides, neither of which algae are exempt from the current EU regulation on novel foods nor do they appear on the China list or European list for approved cosmetic ingredients.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

1-45. (canceled)

46. A modified strain of Chlorella vulgaris having a chlorophyll content in a range of 0.001 to 0.5 mg/g dry cell weight.

47. The modified strain of Chlorella vulgaris of claim 46, wherein the modified strain of Chlorella vulgaris is a heterotroph.

48. The modified strain of Chlorella vulgaris of claim 46, wherein the modified strain of Chlorella vulgaris is obtained from a wild-type strain of Chlorella vulgaris and/or a variation of the wild-type strain of Chlorella vulgaris by performing mutagenesis.

49. The modified strain of Chlorella vulgaris of claim 46, wherein the modified strain of Chlorella vulgaris comprises a mutation at one or more positions in a nucleic acid sequence or an amino acid sequence, and wherein each mutation is one of: a substitution, an insertion, or a deletion relative to a native strain of Chlorella vulgaris, and wherein the modified strain has a chlorophyll content in a range of 0.001 to 0.5 mg/g dry cell weight

50. The modified strain of Chlorella vulgaris of claim 49, wherein the mutation is a transition mutation.

51. The modified strain of Chlorella vulgaris of claim 49, wherein the mutation is in any of a coding region or a non-coding region of the nucleic acid sequence, and wherein mutation in the coding region results in any of: a neutral gene expression, an altered gene expression and/or a modified amino acid sequence.

52. The modified strain of Chlorella vulgaris of claim 49 wherein the modified amino acid sequence comprises codon replacement of one codon for another codon, and wherein the modified codon codes for any of: a chemically similar amino acid, a different amino acid, or a stop codon.

53. The modified strain of Chlorella vulgaris of claim 49, wherein the mutation results in a one or more mutated genes, and wherein the one or more mutated genes is associated with a metabolic change in: a tetrapyrrole biosynthesis pathway, a chlorophyll biosynthesis pathway, a carotenoid biosynthesis pathway, or other pathways.

54. The modified strain of Chlorella vulgaris of claim 49, wherein the one or more mutated gene is associated with at least 90% reduced gene expression relative to the native strain of Chlorella vulgaris, wherein the reduced gene expression result in the chlorophyll content in a range of 0.001 to 0.5 mg/g dry cell weight in the modified strain of Chlorella vulgaris.

55. The modified strain of Chlorella vulgaris of claim 49, wherein the nucleic acid sequence has one or more mutations encoding for amino acid sequences having mutations in magnesium chelatase subunit I (as set forth in SEQ ID NO: 1), O-methyltransferase (as set forth in SEQ ID NO: 2), magnesium-protoporphyrin O-methyltransferase (as set forth in SEQ ID NO: 3), 15-cis-phytoene desaturase (as set forth in SEQ ID NOs: 4 and 5), zeta-carotene desaturase (as set forth in SEQ ID NOs: 6 and 7).

56. The modified strain of Chlorella vulgaris according to claim 46, wherein the modified strain of Chlorella vulgaris has a chlorophyll content lower than a chlorophyll content of the wild-type strain of Chlorella vulgaris from which it is derived, when grown under the same conditions; and optionally wherein the modified strain of Chlorella vulgaris has a chlorophyll content in the range of at least 90% to 99.9% lower than the chlorophyll content of the wild-type strain of Chlorella vulgaris grown under the same conditions.

57. The modified strain of Chlorella vulgaris of claim 46, wherein the reduced chlorophyll content is associated with at least one of: chlorophyll a (α-chlorophyll) and/or chlorophyll b (b-chlorophyll) and collectively, the chlorophyll content is in a range of 0.001 to 0.5 mg/g dry cell weight.

58. The modified strain of Chlorella vulgaris of claim 46, wherein the modified strain of Chlorella vulgaris has a lutein content below 9 mg/g dry cell weight.

59. The modified strain of Chlorella vulgaris of claim 46, wherein the modified strain of Chlorella vulgaris has a minimum protein content of at least 20% w/w.

60. A method of producing a modified strain of Chlorella vulgaris having a chlorophyll content in a range of 0.001 to 0.5 mg/g dry cell weight, wherein the method comprises:
a) obtaining a parental strain of Chlorella vulgaris,
b) performing mutagenesis of the parental strain of Chlorella vulgaris,
c) cultivating the mutated strain of Chlorella vulgaris: at a specific temperature, for a predefined period of time, without presence of light and in the presence of an organic carbon energy source; and
d) identifying colonies of the mutated strain of Chlorella vulgaris having a phenotype different from the parental strain of Chlorella vulgaris as the modified strain of Chlorella vulgaris.

61. The method of claim 60, wherein the parental strain of Chlorella vulgaris is a wild-type strain of Chlorella vulgaris or a variation of a wild-type strain of Chlorella vulgaris and wherein the mutagenesis is performed by exposure of the parental strain of Chlorella vulgaris to a non-lethal quantity of a mutagenic chemical

62. The method of claim 60 wherein mutagenesis is performed by exposure of the parental strain of Chlorella vulgaris to a physical mutagen, wherein the physical mutagen comprises at least one of: UV light, gamma rays, X-rays.

63. The method of claim 60, wherein the identification of the modified strain of Chlorella vulgaris comprises sorting the cells by flow cytometry.

64. The method of claim 60, wherein the specific temperature is in a range of 20 to 35° C.

65. The method of claim 60, wherein the predefined period of time is in a range of 1 to 3 weeks.


SEQUENCE LISTING (8 sequences)

SEQ ID NO: 1 - protein, 413 aa, Chlorella vulgaris
MFTHTAAIRQRCPAGRPRFLVSAVAAPERLKAAKKRPAFPFTRLAGQEDMKLALLLNVVDPSIGGVLIMGDRGTGKSVAVRSLVELLPMIDVVPEDPFNSHPTDPKLMGPYVLDRYAKGEKLPATTMRTPLVELPLGATEDRICGTIDIEKALGEGIKAYEPGLLARANRGILYVDEVNLLDDGLVDVVLDSAAGGLNTVEREGISIVHPAKFIMIGSGNPAEGELRPQLLDRFGLSVNVETLMDVGQRTQMVMDRIAYEASADKFAETVSGDQDELRQKLSAARDLLPKVQVSRELKLKISELCSYLDIDGVRGDITTNKGVRAFCAFEGRTTATKADLERIAPLVLNHRMRKDPLDPIDGGTKVRIALRRLLNPEAAKAEEAKKKKAAEEAAAAAAAENKKKGAWGGLPGR

SEQ ID NO: 2 - protein, 277 aa, Chlorella vulgaris
METRQLKPAAARGRLAVIAAAIMVVLLLSYVLYQLADNDTRLRQLESQAAATAAAAAAHKPMKGSSDQLITTSRQSIQDKPAEPEVKPIPQLNSRTDIAQLLESEGLTTGAELGVLVGDFTEHTLNIWQGCKTYYLVDLWAKQKNYVDLANSDDKEQELRYETTQKRLAPWEGKTQIKRMYTTEAAKQIPDNSLDYVYVDARHDYCGVMEDLQAYWPKLRSGGIFAGHDYIDAHYHALKATNQDWSVCMDGTVNFGAVKGAVNEFAEEHGLTVVATV

SEQ ID NO: 3 - protein, 342 aa, Chlorella vulgaris
MSLSVLTSPTALQQRAQCSARTSRQAPRIVCSAARLAIRSQQLAAGALAAAAALQTAAPALAAVEAVGQVADGASLSLALGGGAAVAALSAALVMTDPQKRRSQQMQETGGNELEAVKKYFSTAGFERWNKIYGETDEVNKVQMDIRNGHAQTVEKVLRWVDEEGGVEGTTICDAGCGTGSLAIPLALRGATVSASDISSSMAGEAQRRYEAAVANGAKAPKVAPKFETLDLESCSGRYNTVCCLDVMIHYPQDKVDGMVQHLASLAEERLIISFAPNTLALTILKAIGELFPGPSKATRVYLHKEDDVEAALARAGFKVTKREMTSTQFYFSRLLEAQRCT

SEQ ID NO: 4 - protein, 551 aa, Chlorella vulgaris
MQSTTMSSAGVSATVCRRQGGFKAAMRGQAVAQQHHARAGRSSLRVVARDFPKPDFEKEKTFQEMAAISAAVKAAPRPKEPLTVVIAGAGLAGLSTAKYLVDAGHKPIVLEARDVLGGKVAAWKDEDGDWYETGLHIFFGAYPNLMNLFKELNIEDRLQWKQHSMIFAVRDSPGEFSRFDFPDLPAPLNGIVAILRNNQMLSWPEKIQFALGLLPAIVYGQPYVEAQDDKTVTEWMVKQGVPARVNDEVFIAMAKALNFIDPDELSMICVLIALNRFLQERHGSKMAFLDGCPPERLCQPMVDYVTARGGEVRMKAGIKNIELNEDGSVKQYNLLSGESITADLYVSAVPVDIFKRLLPAPWYQQQFFSKLDKLVGVPVINIHIWFDRKLTTVDHLLFSRSPLLSVYADMSTTCREYFDTERSMLELVFAPAEKWIGRPDEEIIAATMKELENLFPTEIAADGSKAQIRKYKVVKTPLSVYKTVPECEPCRPTQRTPIRNFYLAGDYTKQRYLASMEGATFSGKLCAQAIAEDWNTSAVKPSQPAKEKALA

SEQ ID NO: 5 - protein, 551 aa, Chlorella vulgaris; identical to SEQ ID NO: 4

SEQ ID NO: 6 - protein, 584 aa, Chlorella vulgaris
MQASAVASPALLQGQAATKAFAPLSQRATGRVRSSRTVRSQAVQAPAKADVSNVSDVALKDVPLRSLFPDVPTIRPGAPKLKVAIVGSGLAGLSTAVELLDQGYEVDIYESRPWIGGKVASFKDKDGNDIEMGLHVFFGCYFNLFRLMAKCGVLENLLLKDHTHTFINKGGDVRELDFRFFLGGKKIGAPFHGLNAFFTTPQLSLTDKVANSLALGTSPIVRSLFDPEGGMRSVRALDNVSFTRWFKSHGGSQASIDRMWDPIAYALGFLNCDDISARCMLSIFQFFATKTDASALRMLNGSPAERLLKPIADYITAKGGRIHTRWGCREIQYEQGPGGQTRVTGLHMTKAGQEQTVTADAYVAALDVPGAKRLLPQAWRSDKFFDNVFQLVGVPVITVQLRYDGWVTEMQDQSKMKDMSGSARGINNLLYSADADFSCFADLALTSPVDYFKQGQGSLMQCVLTPADPYLPKTNEDIAAEVDKQVRRLFPSAAGLTLKWHSVVKISQSLYREAPGVDPFRPDQRTPVPNFFLAGSYTKQDYIDSMEGATLSGRQCAYKVLEDSEQLARAAGAAKQQQQQAVAA

SEQ ID NO: 7 - protein, 584 aa, Chlorella vulgaris; identical to SEQ ID NO: 6

SEQ ID NO: 8 - protein, 527 aa, Chlorella vulgaris
MRPAPCASTTASRAAEASTSGRGAAPQQNHRWRAALRQQNSSSGRRWQSGQQQQLSSALSQRGQRLQAATSPPFFERGSSSLDEAAGGAQLTAASGAVGQERIGVLLLNLGGPDSLDDVQPFLYNLPADPDIIRLPAPAQFLQPVIAQLVSTLRAPKSREGYEAIGGGSPLRRITEEQAAALRASLQSKGISAHTYVAMQYWFPFTEEAISAVKRDGITQLVVLPLYPQFSVSTSGSSLRLLERLLKQDPMLTGVKHVVIPSWYQRPGYVRAMADLIVAELRSGAFPSEELDDVEIFFSAHGVPVSYIDEGDPYKEEMEECVQLVMGELKRRGIANSHTLAYQSRVGPVEWLKPYTDDSIRDLARGGVRSLLAVPISFVSEHIETLEEIDCEYRELAEESGIQHWGRVPALNTNPTFIDDLADAVAEALPYVGSLQRPAGALPLSAGDSLVPLGEVDALLETYDRDRKALPTPVLMWRWGWTKSAETWNGRIAMLALVLILLLEATTGRGVLTLTRMLEMHLMPPPS